(19) United States
(12) Patent Application Publication
(10) Pub. No.: US 2023/0380724 A1
(43) Pub. Date: Nov. 30, 2023

(54) SYSTEM AND METHOD FOR ANALYZING GAIT-RELATED HEALTH AND PERFORMANCE OF AN EQUINE ANIMAL

(71) Applicant: Walkbeat AB, Göteborg (SE)

(72) Inventors: Siddhartha KHANDELWAL, Göteborg (SE); Joao Elias BRASIL BENTES JUNIOR, Göteborg (SE)

(73) Assignee: Walkbeat AB, Göteborg (SE)

(21) Appl. No.: 18/245,924

(22) PCT Filed: Sep. 27, 2021
(86) PCT No.: PCT/SE2021/050939

§ 371 Date: Mar. 20, 2023

(30) Foreign Application Priority Data
Sep. 25, 2020 (SE) 2051119-2

(51) Int. Cl. A61B 5/11; G16H 50/70; A61B 5/00

(52) U.S. Cl. CPC A61B 5/112; A61B 5/1126; G16H 50/70; A61B 5/6828; A61B 2503/40; A61B 2562/0219; A61B 2562/04

(57) ABSTRACT

An analysis system (1) for assessing gait-related health and performance of an equine animal (5) is provided. The analysis system (1) comprises at least a first, second, third and fourth sensor devices (20a, 20b, 20c, 20d) each arranged at one leg of an equine animal (5). The system further comprises a computing unit (10) configured to receive said gait data (22a, 22b, 22c, 22d) from said at least first, second, third and fourth sensor devices (20a, 20b, 20c, 20d), receive at least one metadata (110) associated with the equine animal (5), analyze said received gait data (22a, 22b, 22c, 22d) and/or said received metadata (110) for determining at least one gait parameter (210) related to equine stride characteristics of said equine animal (5), and analyzing the at least one gait parameter (210) and said at least one metadata (110) to assess gait-related health and performance of the equine animal (5).

### TECHNICAL FIELD

The present invention generally relates to the field of analyzing the gait of a moving subject, and more particularly to a system and method for analyzing equine health and performance.

### BACKGROUND

Gait analysis refers to a study of observing animal locomotion provisioned by measuring instruments for measuring body movements and muscle activity. The measurements provided from measuring instruments in a gait analysis study may be used to assess and treat movement impairing conditions. Assessments include for instance classifying movement patterns to determine what and how well an activity is being performed. Low levels of physical activity have been associated with increased risk of chronic diseases and thus knowing which activities an animal or a person performs during a day gives insights into their overall health status. As such, numerous works have been dedicated to classifying daily-living activities using wearable sensors.

Over the years, studies have been dedicated to analyzing gait. These gait measurements relate to spatio-temporal measures such as speed, cadence or step frequency, stance time, swing time and double support time.

In light of the observations above, the present inventors have realized that there is room for improvements when it comes to technical provisions for analyzing gait and/or assessing gait quality.

### SUMMARY

It is accordingly an object of the present invention to mitigate, alleviate or eliminate at least some of the problems referred to above, by providing an analysis system for analysing health and performance of an equine animal.

Other aspects of the invention and its embodiments are defined by the appended patent claims and are further explained in the detailed description section as well as on the drawings.

In a first aspect of the invention, an analysis system for assessing gait-related health and performance of an equine animal is provided. The analysis system comprises: at least a first sensor device arranged at a region of a first leg of the equine animal, a second sensor device arranged at a region of a second leg of the equine animal, a third sensor device arranged at a region of a third leg of the equine animal and a fourth sensor device arranged at a region of a fourth leg of the equine animal, wherein the at least first, second, third and fourth sensor devices each comprise at least one 3-axis accelerometer and at least one 3-axis gyroscope, and wherein said at least first, second, third and fourth sensor devices are configured to provide gait data; and a computing unit configured to: receive said gait data from said at least first, second, third and fourth sensor devices, receive at least one metadata associated with the equine animal, analyze said received gait data and/or said received metadata for determining at least one gait parameter related to equine stride characteristics of said equine animal, and analyzing the at least one gait parameter and said at least one metadata to assess gait-related health and performance of the equine animal.

In one embodiment, the at least one gait parameter comprises information of at least one computed energy density spectrum.

In one or more embodiments, the computing unit is further configured to store said assessed gait-related equine health and performance and/or to communicate said assessed gait-related equine health and performance to an external device having a display, wherein the external device is configured to present said assessed gait-related equine health and performance to a user.

In one or more embodiments, the metadata comprises one or more of: information of subject data of the equine animal, information of person data of persons related to the equine animal, information of accessory data related to accessories of the equine animal, and information of training data of the equine animal.

In one or more embodiments, the metadata is based on data received from at least one additional sensor and/or based on data being inputted to the system by a user.

In one or more embodiments, the at least one additional sensor is one or more of: a GPS-sensor, a temperature sensor, a weather sensor, and a pulse sensor.

In one or more embodiments, the at least one gait parameter comprises one or more of: information relating to stride details of the equine animal, information relating to activity details of a training session of the equine animal, and information relating to gait of the equine animal.

In one or more embodiments, each one of the at least first, second, third and fourth sensor devices further comprises a magnetometer.

In one or more embodiments, each one of the at least first, second, third and fourth sensor devices are arranged in one sensor device holder.

In one or more embodiments, the assessed gait-related equine health and performance is used to detect at least one of: one or more improvements in performance of the equine animal, no or at least one minor change in the performance of the equine animal, no or at least one minor change in the health of the equine animal, and/or an increase in risk of injury and/or lameness of the equine animal.

In one or more embodiments, the assessed gait-related equine health and performance is used to make or suggest changes in said metadata.

In one or more embodiments, the gait parameters and metadata are analyzed by comparing it against one or more baselines and/or against historical data.

In one or more embodiments, the computing unit is further configured to: receive a set of acceleration signals from each sensor device; for each set of received acceleration signals, compute a resultant acceleration signal; based on said computed resultant acceleration signals, determine if the equine animal is performing a gait related activity or is inactive; and if it is determined that the equine animal is performing a gait related activity, compute an accelerometer energy density spectrum for each resultant acceleration signal, wherein each accelerometer energy density spectrum corresponds to one leg of the equine animal.

In one or more embodiments, wherein determining if the equine animal is performing a gait related activity further involves: computing a moving standard deviation signal of the resultant acceleration signals; generating a filtered acceleration signal by performing 1-D morphological filtering of said computed moving standard deviation signal; and determining if a total number of elements of the filtered acceleration signal having a value greater than or equal to a value of a corresponding element of a predetermined walking threshold.

In one or more embodiments, the computing unit is further configured to: receive a set of acceleration signals from each sensor device; for each set of received acceleration signals, compute a resultant acceleration signal; compute an accelerometer energy density spectrum for each resultant acceleration signal, wherein each accelerometer energy density spectrum corresponds to one leg of the equine animal, wherein the computing unit is further configured to: receive a set of gyroscope signals from each sensor device; for each set of received gyroscope signals, compute a resultant gyroscope signal; and for each resultant gyroscope signal, compute a gyroscope energy density spectrum wherein each gyroscope energy density spectrum corresponds to one leg of the equine animal.

In one embodiment, the computing unit is further configured to analyze the computed energy density spectrums by: measuring the variability by comparing each energy density spectrum to itself over a predetermined time period, and/or measuring the symmetry by comparing an energy density spectrum of a left leg of the equine animal to an energy density spectrum of a right leg of the equine animal, and/or measuring the normality by comparing each energy density spectrum to at least one energy density spectrum of a leg from a reference population group exhibiting no gait pathology.

In one or more embodiments, the computing unit is further configured to compute statistical data and/or historical data of at least one of the metadata and the at least one gait parameter.

In one or more embodiments, the computing unit is further configured to generate and transmit a deviating signal to the external device if at least one of the metadata and/or the at least one gait parameter exceeds a predetermined deviating threshold value.

In a second aspect of the invention, a method for assessing gait-related health and performance of an equine animal is provided. The equine animal is equipped with at least a first sensor device at a region of a first leg of the equine animal, a second sensor device at a region of a second leg of the equine animal, a third sensor device at a region of a third leg of the equine animal and a fourth sensor device at a region of a fourth leg of the equine animal, wherein the at least first, second, third and fourth sensor devices each comprise at least one 3-axis accelerometer and at least one 3-axis gyroscope, and wherein the at least first, second, third and fourth sensor devices being configured to provide gait data. The method involves: receiving said gait data from said at least first, second, third and fourth sensor devices; receiving at least one metadata associated with the equine animal; analyzing said received gait data and/or said received metadata for determining at least one gait parameter related to equine stride characteristics of said equine animal; and analyzing the at least one gait parameter and said at least one metadata to assess gait-related health and performance of the equine animal.

The invention described herein has several benefits for different stakeholders in the equine eco-system such as trainers, owners, veterinarians, farriers, saddle fitters, equine therapists, breeders, riding schools and animal R&D centers and universities. For the trainer the system allows to fine tune every aspect of training with gait insights to maximize performance and minimize risk of lameness/injury over the lifetime of the horse. The benefits will now be summarized herein.

One benefit includes that different performance parameters can be used to maximize gait quality and/or other measures depending on the sport, such as speed, jump height and pyramids of training. Moreover, identification of which combination of training factors, e.g. surface/shoe/rider/driver, etc. (metadata) may be provided, which leads to better performance and health over time. The invention furthermore provides users with the opportunity to select the currently top performing horses to compete, which may vary from time to time. Additionally, the user may find and replicate gait signatures (horses), ultimately leading to better performance and healthier horses. Yet additionally, early detection of injuries/lameness can be provided, and the tracking of rehabilitation processes for deciding when to resume training may be provided. For the owner the system and method as claimed herein allows their horse the competitive edge it deserves as they can follow improvements over time. The owner will also be able to communicate better with trainers and veterinarians with the help of long-term gait information. The owner can also conduct quick gait tests during buying and selling. As such, additional advantages and effects involve following and understanding your horses' development during training, increasing the transparency during a purchase, trade or sell of a horse, early intervention when gait abnormalities are detected, clarity around horse care and rehabilitation, and the avoidance of injuries and cases of lameness.

Moreover, the system and method as claimed herein can also be beneficial for riding schools. It will help the students to improve with immediate feedback on their riding style and make comparisons over time. The system can educate the students with objective information on Pyramids of Training for different gaits as well as increasing their interest in riding with interactive sessions. As such, some benefits include immediate feedback to the rider which allows for quick response and interactive learning, and also the decrease in risk of horse injuries as caused by, for instance, an improper riding technique.

The system and method as claimed herein is furthermore beneficial for veterinarians. They can conduct fast gait tests with walking and trotting in a straight line, lunging left and right to detect even minor deviations, often in response to stress tests, joint blocks or other interventions/treatments, which are difficult to catch with the naked eye. The veterinarians will have a tool to communicate with their clients using objective gait information as a basis during rehabilitation and recovery. The history of gait information can be used to improve future diagnosis. As such, benefits provided for the veterinarian may involve support in diagnosis based on current gait quality and history, development of injury/rehabilitation during follow-ups, and following, tracking and prescribing custom rehabilitation based on the horse's initial response to medication, diagnosis or treatment. Moreover, benefits involve the sharing of objective analysis with horse owners for traceability and digital rehabilitation which can be used for future services.

A farrier, which is a specialist in equine hoof care, including the trimming and balancing of horses' hooves and the placing of shoes on their hooves, can also benefit from the system described herein. They can conduct fast gait tests before and after shoeing to make objective evaluation of shoeing quality. The system and method as claimed herein give them a tool to fine tune the shoeing process and technique to get the best performance from the horse. The farrier will have a tool to communicate with their clients using objective gait information as a basis; and conduct follow-ups. The history of gait information can be used to improve future shoeing. As such, the benefits provided for the farrier may involve providing clarity around hoofcare and rehabilitation, quantification of shoeing techniques for horse performance, traceability and a communication tool for customer relations, and tracking and adapting of shoeing plans to ensure maximal gait quality.

The system and method as claimed herein are also beneficial for researchers and animal R&D facilities in different fields. The system allows to collect precise, accurate movement data with time-synchronised inertial sensors with that have global timestamps. The system will promote collaboration as well as conducting research on-the-go at remote locations with easy-to-manage database. As such, the benefits include, but are not limited to accessing data collections in remote locations, conducting extensive studies in the real world to open up new strains of research, information and learnings, and accessing all levels of information which ensures a wider sample size and generalization of research to all horses.

Finally, the system and method as claimed herein provides breeders the opportunity to use gait history and metadata to breed horses for specific sports and disciplines, develop the horse's overall quality and personality based on objective gait quality measures. Moreover, transparency during a purchase, trade or sell of a horse based on objective gait history and quick gait tests is provided.

It should be emphasized that the term “comprises/comprising” when used in this specification is taken to specify the presence of stated features, integers, steps, or components, but does not preclude the presence or addition of one or more other features, integers, steps, components, or groups thereof. All terms used in the claims are to be interpreted according to their ordinary meaning in the technical field, unless explicitly defined otherwise herein. All references to “a/an/the [element, device, component, means, step, etc]” are to be interpreted openly as referring to at least one instance of the element, device, component, means, step, etc., unless explicitly stated otherwise. The steps of any method disclosed herein do not have to be performed in the exact order disclosed, unless explicitly stated.

### BRIEF DESCRIPTION OF THE DRAWINGS

Objects, features and advantages of embodiments of the invention will appear from the following detailed description, reference being made to the accompanying drawings, in which:

FIGS. 1a-b are schematic illustrations of a non-limiting example of an analysis system in which embodiments of the present invention may be exercised.

FIG. 2 is a schematic illustration of an analysis subject in one embodiment seen from a top view perspective.

FIG. 3 is a schematic block diagram of a sensor device holder used for analysis in one embodiment.

FIGS. 4a-b are schematic block diagrams illustrating the basic internal hardware and software layout of a mobile communication terminal according to embodiments of the invention.

FIG. 5 is a schematic block diagram illustrating features forming part of assessing equine health and performance according to embodiments of the invention.

FIG. 6 is a schematic block diagram illustrating features forming part of assessing equine health and performance according to one embodiment.

FIGS. 7a-h are illustrations of different gait parameters according to embodiments of the invention.

FIGS. 8a-c are block diagrams illustrating procedural steps of assessing equine health and performance using metadata and/or gait parameters according to embodiments of the invention.

FIG. 9 is a flowchart of a part of a method of assessing equine health and performance according to embodiments of the invention.

FIGS. 10a-e are flowcharts of parts of a method of assessing equine health and performance according to embodiments of the invention.

FIG. 11 is an illustration of an external device generally according to some embodiments of the invention.

FIG. 12 is a flowchart illustrating feedback loops when assessing gait quality according to one embodiment.

### DETAILED DESCRIPTION

Embodiments of the invention will now be described with reference to the accompanying drawings. The invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the particular embodiments illustrated in the accompanying drawings is not intended to be limiting of the invention. In the drawings, like numbers refer to like elements.

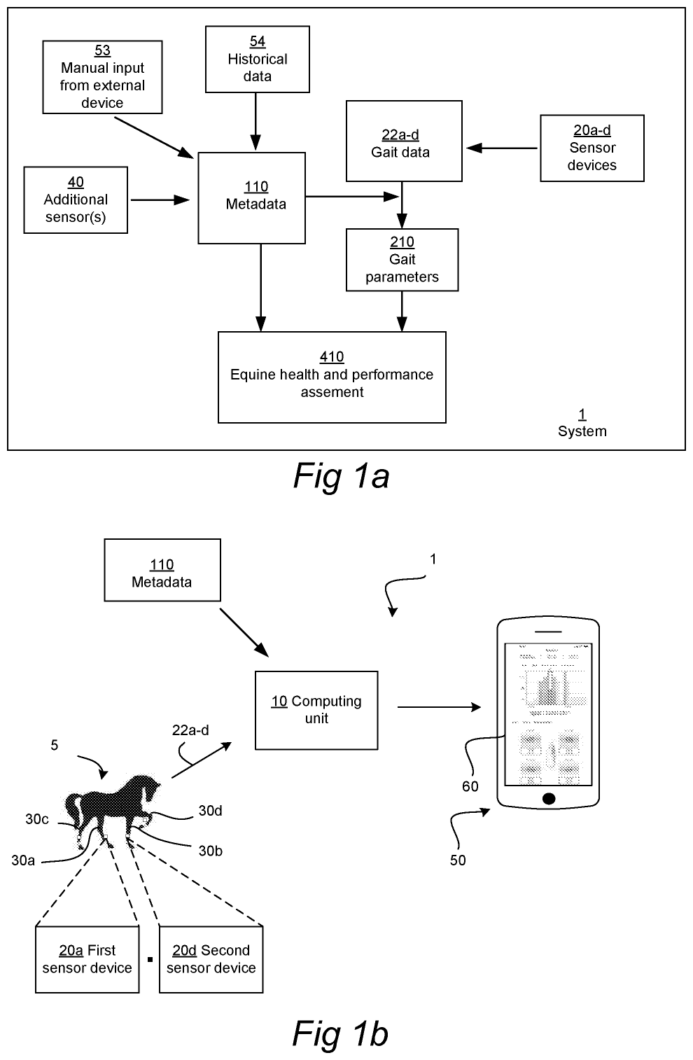

FIGS. 1a-b illustrate an equine health and performance analysis system 1 generally according to an embodiment of the present invention.

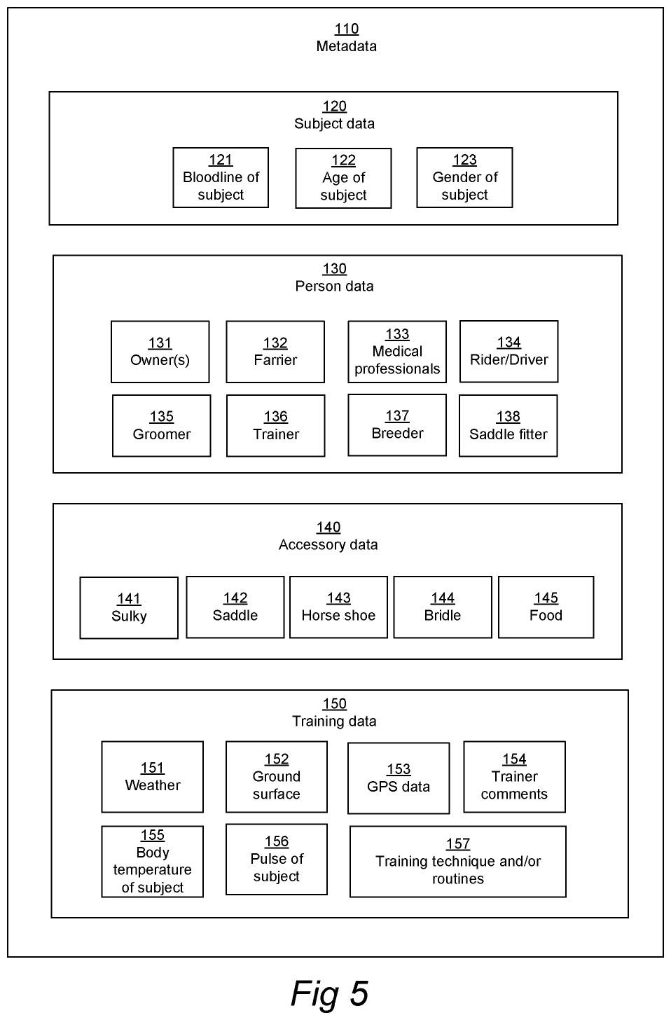

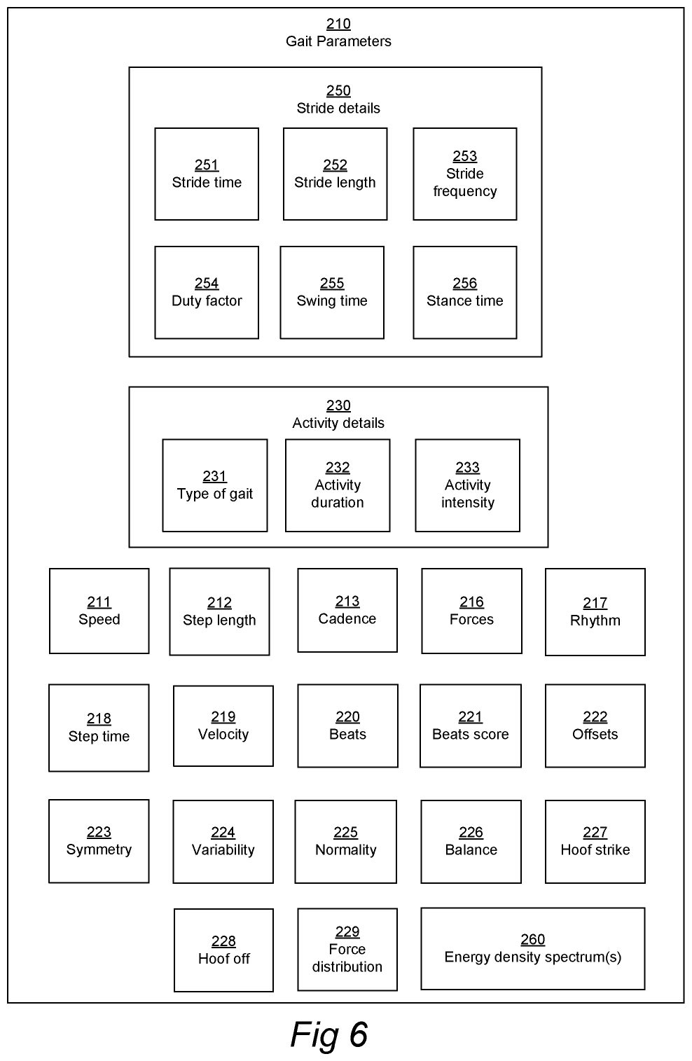

The system 1 comprises a plurality of sensor devices 20a-d that are configured to collect respective gait data 22a-d of the subject 5. The gait data 22a-d is evaluated and analyzed to generate gait parameters 210, which will be described more in detail with reference to FIG. 6, that are used to assess the quality of the gait. Metadata 110 relating to the subject 5 and its environmental factors, described more in detail with reference to FIG. 5, is collected manually by a user 53, automatically by the system 1 itself, historical data 54, and/or by one or more additional sensors 40. Additional sensors 40 could be a pulse sensor, a temperature sensor, a weather sensor and/or a GPS.

The system 1 comprises one or more subjects 5 being subjects for gait analysis. In the exemplifying embodiment as illustrated by FIG. 1b, an equine animal 5 is being analyzed. The information described throughout the present disclosure will be directed at equine animals 5. In alternative embodiments, a subject 5 may be for instance a primate animal, a feline animal, a canine animal, or any other animal family suitable for being subjects in gait analysis. The terms “subject” and “equine animal” will be used interchangeably throughout this disclosure, but are both referring to the same subject, i.e. the equine animal 5 (such as the one shown in FIG. 1b).

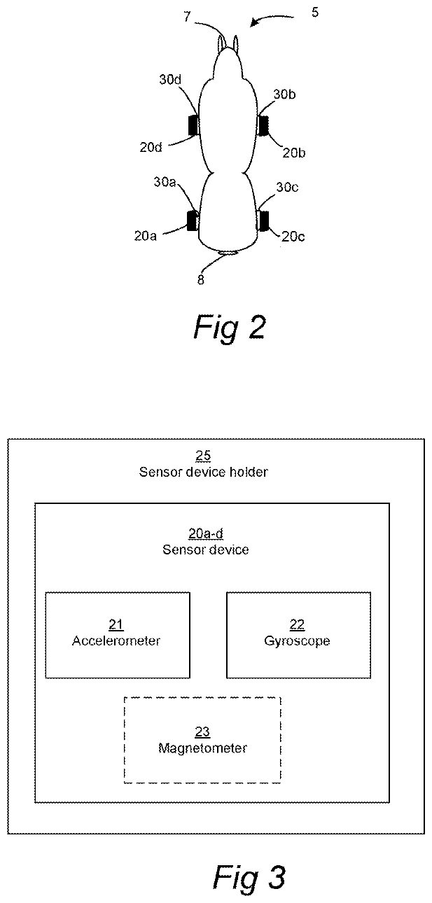

The analysis system for assessing health and performance 1 further comprises at least a first and a second gait sensor devices 20a, 20b configured to provide gait data 22a, 22b of the equine animals 5. In one preferred embodiment, the system 1 further comprises a third and a fourth gait sensor device 20c, 20d configured to provide gait data 22c, 22d of the equine animal 5. This is shown in FIG. 2. This is preferred if the subject 5 has four legs, as is the case with equine animals 5. In yet other embodiments, the system 1 may comprise an arbitrary number of sensor devices 20a-d positioned on different body parts and configured to store and retrieve gait data 22.

Throughout the present disclosure, it is described that gait data 22a-d is received from a respective sensor device 20a-d. This is referring to that each sensor device 20 is configured to provide one or more bits or streams of gait data 22, for one leg 30 each, of the equine animal 5.

Each sensor device 20 may be arranged at a location suitable for providing accurate gait data 22 of the equine animal 5. For instance, the sensor devices 20 may be arranged at a fetlock region, cannon region, pastern region or at the hoof of a leg 30 of the equine animal 5. As know by a person skilled in the art, the fetlock region is the joint joining the pastern and the cannon region.

As seen in FIGS. 1a-b, the gait analysis system 1 further comprises a computing unit 10. The computing unit 10 may be a cloud-computing unit 10 being included in a distributed cloud network widely and publicly available, or limited to an enterprise cloud. For instance, cloud-computing technologies include, but are not limited to Amazon EC2, Google App Engine, Firebase or Apple iCloud. The computing unit 10 is at least configured to receive gait data 22a-d from the sensor devices 20. Further, the computing unit 10 is configured to analyze said received gait data 22a-d for assessing gait quality relating to the gait characteristics of the equine animal 5. In a preferred embodiment, received gait data 22a-d is analyzed to assess equine gait quality, wherein the equine gait quality is related to equine stride characteristics of an equine animal 5. Moreover, the computing unit 10 may further be configured to receive and analyze metadata 110. The computing unit 10 may further be configured to perform an equine health and performance assessment 410 based on the metadata 110 and the gait parameters 210.

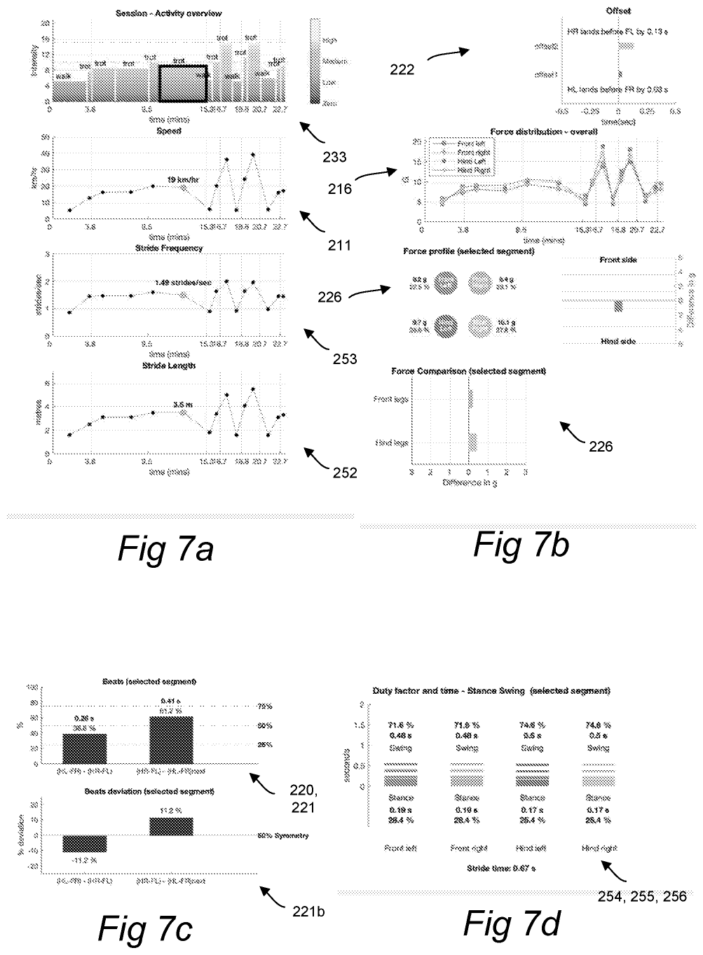

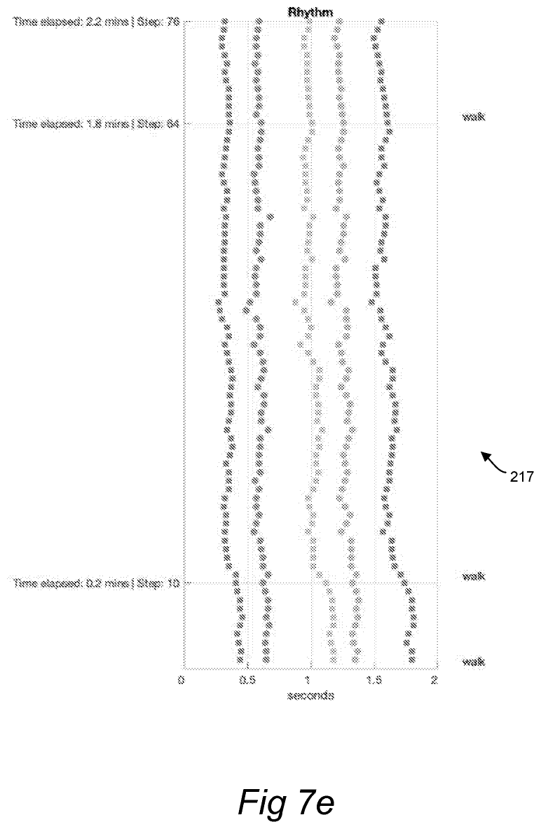

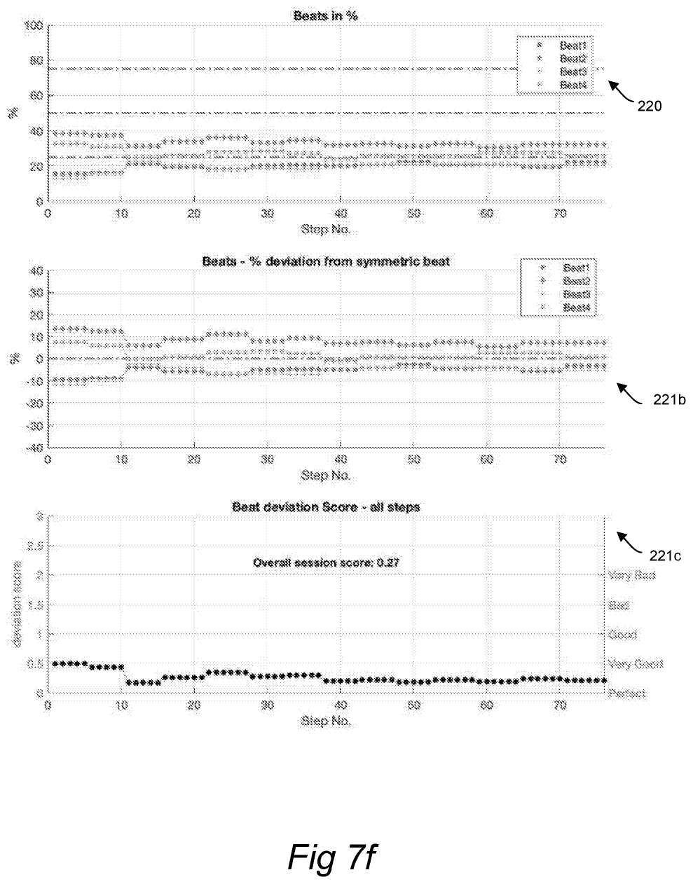

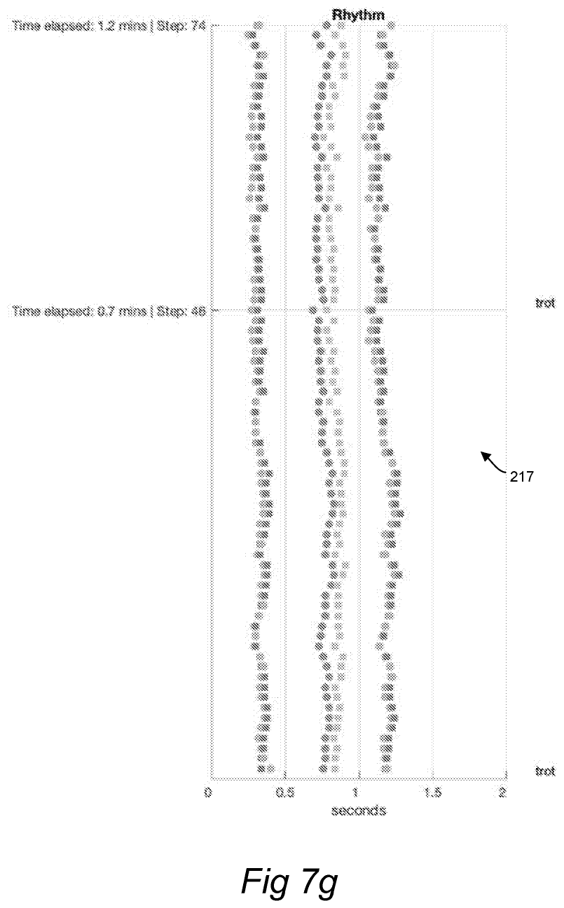

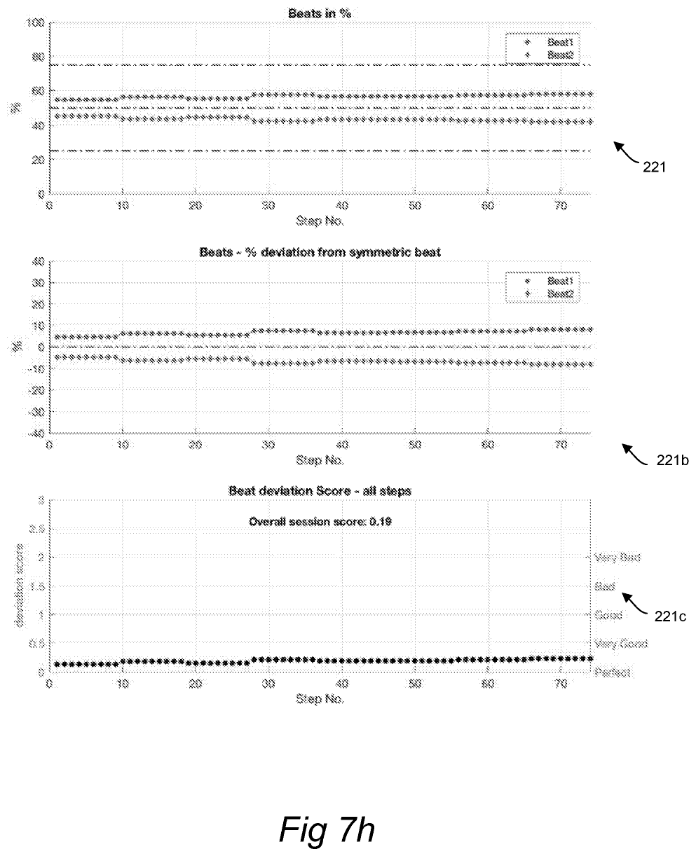

Additionally, the computing unit 10 may also be configured to store the metadata 110 and gait history for long-term analysis, as is described more in detail with reference to FIG. 7a-c.

The computing unit 10 is further configured to communicate the assessed equine health and performance assessment 410 to an external device 50. The external device 50 may be embodied as a mobile terminal, for instance a mobile phone, laptop computer, stationary computer or a tablet computer. Preferably, the external device 50 has a display 60. The display 60 may be a touch screen display or a non-touch screen. The display 60 is configured to present information of the analysis performed by the computing unit 10 and/or the analysis performed by the external device 50. Preferably, the external device 50 is configured to present the assessed equine health and performance information. As will be discussed more in detail later on, this information may be presented as different graphs and/or different values (such as a score, index value, etc.). It should be noted that the analysis performed in the computing unit 10 also could instead be partly or fully performed in the external device 50.

One embodiment of a system and the equine animal 5 is illustrated in FIG. 2, and it is seen from a top view perspective. The equine animal 5 in FIG. 2 has a front part 7 and a back part 8 and four legs 30a-d. For each leg 30a-d, a sensor device 20a-d is arranged at its fetlock, pastern or cannon region. Hence, a first sensor device 20a is arranged at a fetlock region of a first leg 30a (back left leg), a second sensor device 20b is arranged at a fetlock region of a second leg 30b (front right leg), a third sensor device is arranged at a fetlock region of a third leg 30c (back right leg), and a fourth sensor device 20d is arranged at a fetlock region of fourth leg 30d (front left leg). The front left leg will from hereon be noted as FL, the front right leg will from hereon be noted as FR, the hind (back) left leg will from hereon be noted as HL and the hind (back) right leg will from hereon be noted as HR.

As illustrated in FIG. 3, the gait sensor devices 20a-d may be attached to a subject using a sensor device holder 25. The sensor device holder 25 may be any attachment means such as for example an adhesive material or a strap, belt, harness, band or similar. In one embodiment, the gait sensors are arranged in a sensor device holder 25 that is attached to a sensor device 20 to an associated equine leg protection (not shown). The sensor device holder 25 has a first side and a second side, the first and second sides having hook and/or loop structures configured to be attached to corresponding hook or loop structures provided on an associated equine leg protection. An opening is further arranged between the first side and the second side, the opening being configured to receive a sensor device 20.

Equine leg protection, such as open front boots, splint boots or cross county boots, are provided with hook and loop fasteners used to fasten the leg protection around the leg of the equine animal. A sensor device holder 25 as describe above has the advantage of comprising a first side and a second side having either one of a hook or loop structure so that the sensor device holder 25 comprising the sensor device 20, can easily be fastened in the already existing fastening means of the equine leg protection. Thus, no additional fastening means to fasten the sensor device 20 to the leg protection is needed and the equine animal will not be affected or disturbed by the attachment of the sensor device 20. When fastening the sensor device holder 25 in the fastening means of equine leg protection, the sensor device holder 25 and the sensor device 20 are hidden inside the fastening means, thus the equine animal cannot kick away the sensor device 20 and does not risk to be injured by abrasions.

FIG. 3 further illustrates an exemplified embodiment of a sensor device 20a-d. Each sensor device 20a-d preferably comprises at least one accelerometer 21, and at least one gyroscope 22. In a preferred embodiment, the sensor device 20a-d further comprises at least one magnetometer 23. In one embodiment, each sensor device 20a-d comprises at least one 3-axis accelerometer 21, at least one 3-axis gyroscope 22 and at least one magnetometer 23 configured to provide gait data 22a-d. The magnetometer 23 may be a 3-axis magnetometer. As will be described more in detail with reference to FIG. 5, gait data 22a-d may be locally stored, retrieved continuously or at a predetermined timely basis. In one embodiment the provided gait data 22a-d include a set of acceleration signals aₓ, a_{y}, a_{z} retrieved from at least one 3-axis accelerometer 21, set of gyroscope signals gₓ, g_{y}, g_{z} retrieved from at least one 3-axis gyroscope 22. The gait data may further comprise a set of magnetometer signals mₓ,m_{y},m_{z} retrieved from at least one 3-axis magnetometer 23. Accordingly, the sets of acceleration, gyroscope, and magnetometer signals may be included in the gait data 22a-b.

The magnetometer 23 measures the magnetic field or magnetic dipole moment. The magnetometer 23 may measure the direction, strength and/or relative change of a magnetic field at a particular location. In one embodiment the magnetometer is a vector magnetometer that can measure one or more components of the magnetic field electronically. In one embodiment, the magnetometer 23 is a scalar magnetometer that measure the total strength of the magnetic field to which it is subjected, and not its direction. In one embodiment, the at least one magnetometer 23 is used in conjunction with a 3-axis accelerometer to produce orientation independent accurate compass heading information.

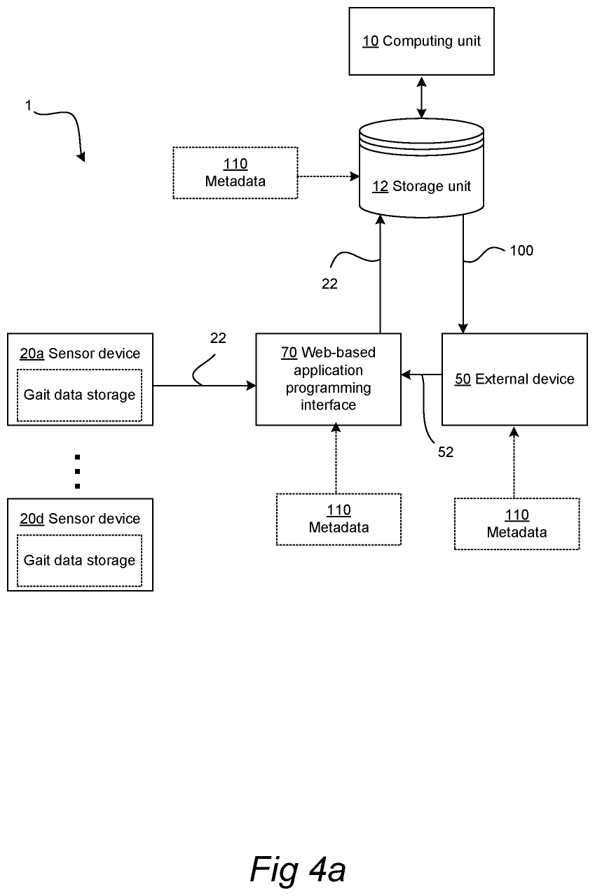

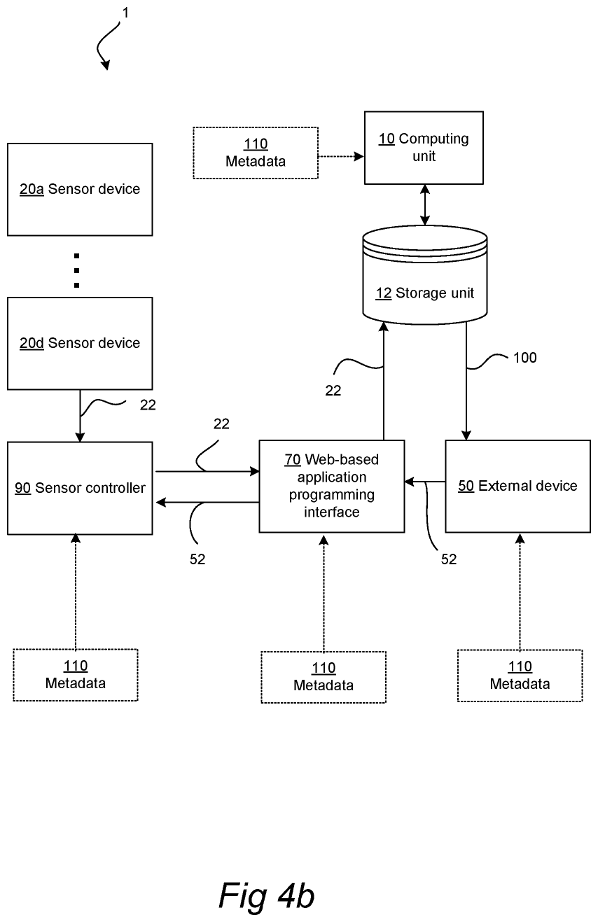

FIG. 4a illustrates a schematic block diagram with the basic internal hardware and software layout of an equine health and performance analysis system 1 according to one embodiment. In addition to the elements described with reference to FIG. 1b, the system 1 may further comprise a storage unit 12 and a web-based API (Application Programming Interface) 70. The web-based API 70 is configured to receive an event request 52 from the external device 50, instructing the web-based API to initiate a gait analysis event. The web-based API 70 is further configured to activate the sensor devices 20 for providing gait data 22 to the web-based API 70 using short-range communication technologies. Examples of such technologies are short-range standards IEEE 802.11, IEEE 802.15, ZigBee, WirelessHART, WIFI and Bluetooth® to name a few. It should be noted that, as is commonly known, the web-based API is arranged to communicate according to more than one technology and many different combinations exist. Further, peer-to-peer connection between the web-based API 70 and the external device 50 may be established using protocol standards such as for instance HTTP, HTTPS, WebRTC, QUIC, IPFS.

Moreover, communications may also be based on transferring data via IoT-services (Internet of Things). In different embodiments of the invention, different IoT-protocols may be utilized. For instance, protocols include, but are not limited to Bluetooth®, WiFi, ZigBee, MQTT IoT, CoAP, DDS, NFC, AMQP, LoRaWAN, RFID, Z-Wave, Sigfox, Thread, EnOcean, celluarly based communication protocols, or any combination thereof.

The storage unit 12 may be run on a cloud-computing platform, and connection may be established using DBaaS (Database-as-a-service). For instance, the storage unit 12 may be deployed as a SQL data model such as MySQL, PostgreSQL or Oracle RDBMS. Alternatively, deployments based on NoSQL data models such as MongoDB, Hadoop or Apache Cassandra may be used. DBaaS technologies include, but are not limited to Amazon Aurora, EnterpriseDB, Oracle Database Cloud Service or Google Cloud. Preferably, the storage unit 12 is deployed on the same platform as the computing unit 10 deployment.

As indicated in dashed lines, gait data 22a-d may be stored locally in the sensor units 20a-d. The gait data 22a-d may be stored locally in the sensor units before being transmitted to a web-based application programming interface 70 or being directly transmitted to the storage unit 12. The computing unit 10 then computes gait parameters 210.

Metadata 110 could be received to the system 1 by either the web-based application programming interface 70, the external device 50, or by the storage unit 12.

In FIG. 4b, a schematic block diagram illustrating another embodiment of the invention is presented. Herein, the gait analysis system 1 further comprises a sensor controller 90 configured to receive the event request 52 from the external device 50 via the web-based API. The sensor controller 90 further comprises an activator application which is configured to control the activation of the sensor devices 20. The activation may be performed automatically as a response to having received an event request 52. Alternatively, the activation may be performed manually by a user. Similar to the embodiment discussed when referencing FIG. 4a, the sensor controller 90 comprises a communication interface based on any short-range communication technology as mentioned. The activator application may for instance be embodied as a mobile application or a web-based application, configured to respond to user input using e.g. physical buttons, touch screen functionalities, audible input, sensorial input, or any combination thereof.

As shown in FIG. 4b, some kinds of metadata 110 that are originating from at least one additional sensor 40, can be stored and processed in the sensor controller 90. This may for example be the case with metadata such as weather data, GPS data, pulse data or temperature data. Although not shown, the system 1 may further comprise means for providing secure communication between software and hardware components of the system. In order to ensure secure communication, messages may be encrypted, encoded, enciphered using a variety of cryptographic hash functions. For instance, SHA-1, SHA-2, CRC32, MD5, or any other commonly used hash function may be used.

The following is an example embodiment of the process of generating a health and performance assessment in an analysis system 1 generally according to the present invention shown in FIG. 4b. The required information to start a new analysis is initiated by the user of the external device 50. The user may activate a trial event by requesting an event request 52 to the web-based API. This may be done using for instance a phone app, a tablet app, a web service or similar, installed on the external device 50. Consequently, the web-based API receives the event request 52, and routes it to the sensor controller 90. The sensor controller 90 triggers a sensor activation signal, which may be communicated via a short-range communication standard. The communication may also be performed using an IoT-service as discussed above. In response to the activation signal from the sensor controller 90, each sensor device 20a-d is configured to stream gait data 22a-d in the form of acceleration data, gyroscope data and magnetometer data to the sensor controller 90 via short-range communication standards. The data may also be transmitted via IoT-services.

When the sensor controller 90 has received a set of acceleration data, gyroscope data and magnetometer data corresponding to a predetermined quantity, the web-based API 70 receives the sets from the sensor controller 90. For instance, the sensor controller 90 may receive approximately 10 seconds of raw data retrieved by the sensor devices 20a-d. If the sensor devices 20a-d are configured to a sampling frequency in hertz, e.g. 128 hertz, the sensor controller 90 may receive approximately 1300 data points of raw data. The web-based API 70 is configured to transmit the retrieved sets of acceleration data, gyroscope data and magnetometer data to the storage unit 12 using for instance a DBaaS-technology as described above.

Subsequently, the computing unit 10 reads the data from the storage unit 12, performs the gait analysis to generate gait parameters 210. The gait parameters 210 are used alone or together with metadata 110 in order to gain a quality and health assessment. This assessment may be transmitted back to the storage unit 12 which stores the received analysis and transmits it to the external device 50.

Attention is now directed towards FIGS. 5 to 11. Herein, analysis system properties and methods are provided for assessing the health status and performance of the equine animal 5. The examples which will be covered are directed towards assessing equine health and performance of an equine animal 5. However, as previously pointed out, the assessment is not restricted to equine animals; the technical provisions may also be suitable for assessing gait quality of other subjects 5 having at least two legs, and preferably four legs.

Before turning into details of how the data is computed, evaluated and used, the details of the terminology metadata 110 and gait parameters 210 will be described with reference to FIGS. 5 and 6. Both the metadata 110 and the gait parameters 210 affect the quality of gait, and thus subsequently affect the health and performance of the equine animal 5.

A schematic illustration of the details of metadata 110 are illustrated in FIG. 5. The metadata 110 should be seen as parameters that can be categorized into different categories. In the example illustrated in FIG. 5 the metadata 110 is categorized into four different categories, but the present disclosure is not limited to the categories illustrated in FIG. 5. Further categories of metadata 110 than the categories illustrated in FIG. 5 can exist. In the exemplary embodiment the data is presented for a horse, however it should be noted that it could be applicable to other animals as well.

In FIG. 5, a first category of metadata 110 is related to subject data 120, a second category is related to person data 130, a third category is related accessory data 140 and a fourth category is related to training data 150. These categories will now be described in more detail. The first category related to subject data 120 comprises information relating to the subject (such as a horse) bloodline or pedigree 121, age of the subject 122 and gender of the subject 123. Metadata 110 in the category subject data 120 is not limited to the subject data 120 listed, other types of subject data 120 can also exist in this category.

In the embodiment where the subject 5 is a horse, the category horse bloodline 121 may comprise a pedigree, the breed history, the female family of the horse, the male family of the horse, previous diseases, DNA-data and other information relating to the history of the horse. The category horse age 122 comprises information about the age of the horse, such as the number of years and/or months. The category horse gender 123 preferably comprises information about the gender, and possibly also about if the horse is castrated or not. Hence, the horse gender 123 category may include information if the horse is a colt, gelding, stallion, filly or mare.

The second category of metadata 110 relates to person data 130. In the category person data 130 there are metadata 110 such as owner(s) 131, farrier 132, medical professionals 133, rider/driver 134, groomer 135 and trainer 136, breeder 137, saddle fitter 138. It should be understood that all kinds of people data that are related to the equine animal 5 could be part of this section.

The third category related to accessory data 140 may comprise information relating to accessory that the equine animal 5 may use. Such accessories may for example be one or more of a sulky 141, saddle 142, horseshoe 143, bridle 144 and food 145. The food 145 may include type of food (such as brand and/or ingredients) and/or the amount of food. The information may further include the time for each delivery of food (such as morning, before training, etc.). Metadata 110 in the category accessory data 140 is not limited to the accessory data 140 listed, other types of accessory data 140 can also exist in this category.

A fourth category is related to training data 150 and comprise information relating to a training session. The training data may for example be one or more of weather 151, ground surface 152, GPS data 153, body temperature 155 of the equine animal 5, pulse 156 of the equine animal 5, training techniques and routine 157, trainer comments 154 as well as other training related parameters. Training data may comprise information regarding training method, training regime, training style, training knowledge and training routine. The weather data 151 may contain information regarding temperature, wind, sun, clouds and so on. The weather data 151 may be collected from a cloud information system originating from weather stations or be gathered from weather sensors. The GPS data 153 may be collected from one or more additional sensors 40, such as a GPS-sensor. The information relating body temperature 124 and/or pulse 125 may be received from one or more additional sensors 40. The additional sensors 40 may for example be temperature sensors, pulse sensors or health sensors configured to measure temperature and/or pulse. The metadata 110 originating from an additional sensor 40 may be referred to as sensor based metadata 110. Hence, weather, GPS, pulse and/or body temperature may be seen as sensor based metadata 110.

Now turning to FIG. 6 illustrating a block diagram of gait parameters 210 that are determined at least based on the gait data 22a-d received from the gait sensor devices 20a-d according to an exemplary embodiment of the present disclosure. In the example illustrated in FIG. 6 the gait parameters 210 are categorized into different categories, but the present disclosure is not limited to the categories illustrated in FIG. 6. Further categories of gait parameters 210 than the categories illustrated in FIG. 6 can exist. In the exemplary embodiment the data is presented for a horse, however it should be noted that it could be applicable to other animals as well.

Gait parameters 210 are related to stride characteristics of the subject 5. Stride characteristics may comprise any type of information associated with the human locomotion, i.e. a pattern of limb movements. Information associated with the human locomotion may, for instance, be retrieved as the gait data 22a-b by the at least first and second sensor devices 20a-b and further analyzed by the computing unit 10. As such, the stride characteristics may comprise one or more energy density spectrums computed from the gait data 22a-b. This will be thoroughly discussed later on.

The gait parameters 210 may comprise information relating to activity details 230. Activity details 230 may comprise information such as type of gait 231, activity duration 232 and/or activity intensity 233. The type of gait 231 may for a horse be walk, trot, right canter, left canter, gallop, tölt, pace, paso fino and trocha. The activity duration 232, or training time, is the time which the activity lasts, for example measured in seconds or minutes. The activity intensity 233 may be measure as “low”, “medium” and “high” and the definition may be based on stride details 250.

The gait parameters 210 may comprise information relating to stride details 250. Stride details 250 are related to a single leg. Stride details 250 may for example comprise information about stride time 251, stride length 252, stride frequency 253, duty factor 254, swing time 255 and/or stance time 256.

Stride time 251 is the time between two consecutive hoof strikes by the same leg, also known as one complete gait cycle. This is usually expressed in seconds. Hence the stride time 251 may be seen as the sum of stance time and swing time. Stride length 252 is the distance covered between two consecutive hoof strikes or hoof offs. This is either measured directly or is computed as the equal to the product of stride time and speed. The stride length is usually expressed in foot or meters. Stride frequency 253 is the number of strides taken in a given time, this is usually expressed as strides per second or Hz.

The duty factor 254 is the ratio of stance time and stride time. The duty factor is expressed as either a fraction between 0 and 1 or as a percentage between 0% and 100%. The swing time 255 is the time a hoof/leg is in the air/not in contact with the ground during one complete gait cycle. This is usually expressed in seconds. The stance time 256 is the time a hoof is in contact with the ground during one complete gait cycle. This is usually expressed in seconds.

The gait parameters 210 could also be one or more of the following: speed 211, step length 212, cadence 213, step time 218, velocity 219, force within gait cycle 216, beats 220, beats score 221, offsets 222, rhythm 217, hoof strike 227, balance 226, symmetry 223, variability 224 and normality 225.

The step time 218 is the time between two consecutive hoof strikes, expressed usually in seconds. The step length 212 is the length of two consecutive hoof strikes. The cadence 213 is number of steps taken in a given time, usually steps per minute. The speed 211 is distance covered by the center of mass of the equine animal in a given time. The speed 211 is either measured directly or computed as the equal to the product of stride length and stride frequency. The speed 211 is usually expressed as km/hr or m/s. The velocity 219 is speed 211 with a heading or specified direction. Force within gait cycle 229 is force experienced by the sensor positioned at the cannon, pastern, fetlock, hoof of each leg during different phases of one complete gait cycle, such as hoof strike, stance, mid-stance, hoof-off, swing, mid-swing. This is usually expressed in Newton or g.

The beats 220 is the time interval between consecutive hoof strikes of all legs, for a given gait type. An example of beats 220 is the following: walk has four beats in the order: HL-FL-HR-FR-the next HL, whereas trot has two beats (FL,HR)-(FR,HL)-(next FL, next HR), and canter has 3 beats, and so on. Beats score 221 is the ratio of beats and stride time. The beats score 221 is expressed as either a fraction between 0 and 1 or as a percentage between 0% and 100%

The rhythm 217 is the uniformity and consistency of beats 220. The rhythm 217 is measured as the deviation from perfect beats for a given gait type. For example, as walk is a 4 beat gait, the perfect beat score is a recurring 25%-25%-25%-25% of all beats for every stride. Rhythm 217, expressed as a number between 0 and 1 or as a percentage is the deviation from perfect beat score, over time.

The offset 222 is the time interval/difference between hoof strikes of legs that are expected to contact the ground simultaneously for certain gaits. For example in trot, diagonal legs are expected to touch the ground simultaneously. This difference can be expressed as positive or negative to indicative which leg landed before or after the other.

The hoof strike 227 is the moment when the hoof (full or in part) makes contact with the ground. Hoof off 228 is the moment when the hoof leaves contact with the ground.

Symmetry 223 is the ratios of parameters that compare left and right side of the body. One example is the ratio of forces exerted by FL and FR, HL and HR. Another example is ratio of beats 220 or rhythm 217 during symmetric gaits such as trot. Variability 224 is the deviation of parameters for each leg or the equine animal as a whole, when compared to themselves, over time. Hence, gait variability 224 is the phenomenon of having changes in gait parameters 210 from one stride to the next. Normality 225 is the deviation of parameters for each leg or the equine animal as a whole, when compared to a normal population, over time.

Balance 226 is the overall force profile that takes into account the differences in the front and hind side of the body; and left and right side of the body.

The gait parameters 210 may be assessed based on its average value, as well as on its minimum and maximum value. The gait parameters 210 may be used alone or together when analysing the gait quality and thus also the health and performance of the equine animal 5.

Some of the gait parameters 210 are assessed using one or more metadata 110. In one embodiment, some of the gait parameters 210 are assessed using sensor based metadata 110, such as for example a GPS-signal. In other embodiments, the gait parameters 210 are based solely on the gait data 22a-d provided by the sensors 20a-d. In yet one embodiment, the gait parameters 210 are based on the gait data 22a-d together with metadata 110 that is inputted by a user. In one embodiment, the gait data is used together with GPS-data in order to gain more accurate information relating to gait parameters regarding position and velocity. However, it should be noted that no GPS-signal, or other sensor based metadata 110, is essential in order to determine gait parameters 210.

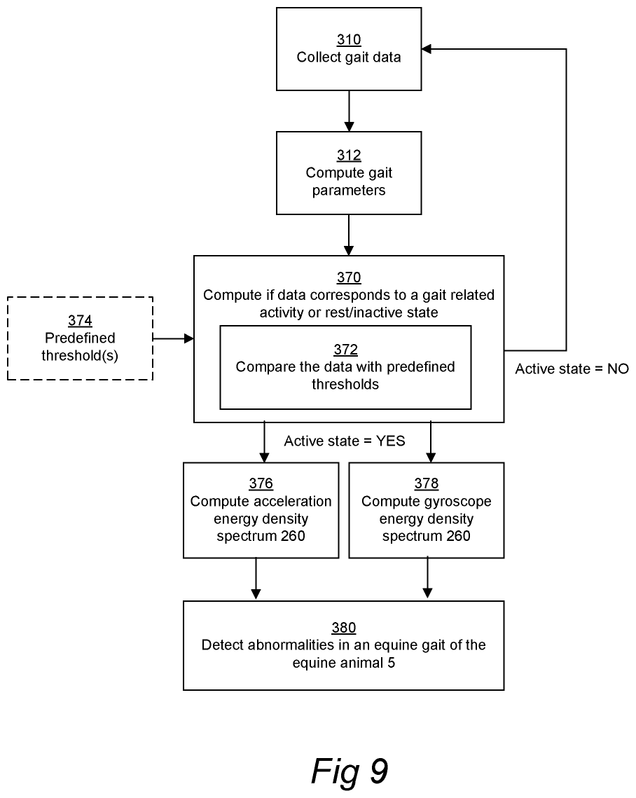

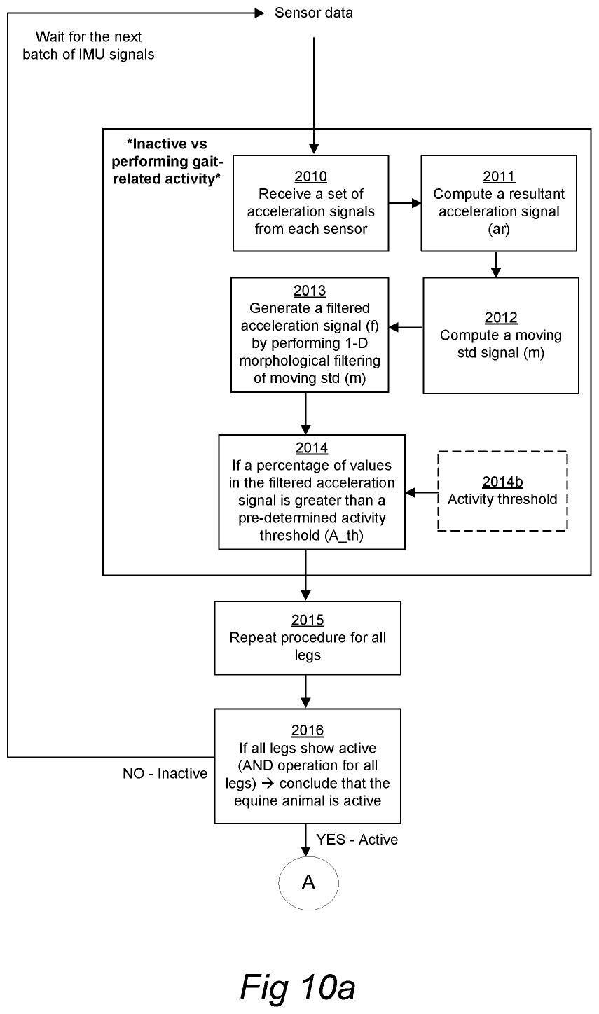

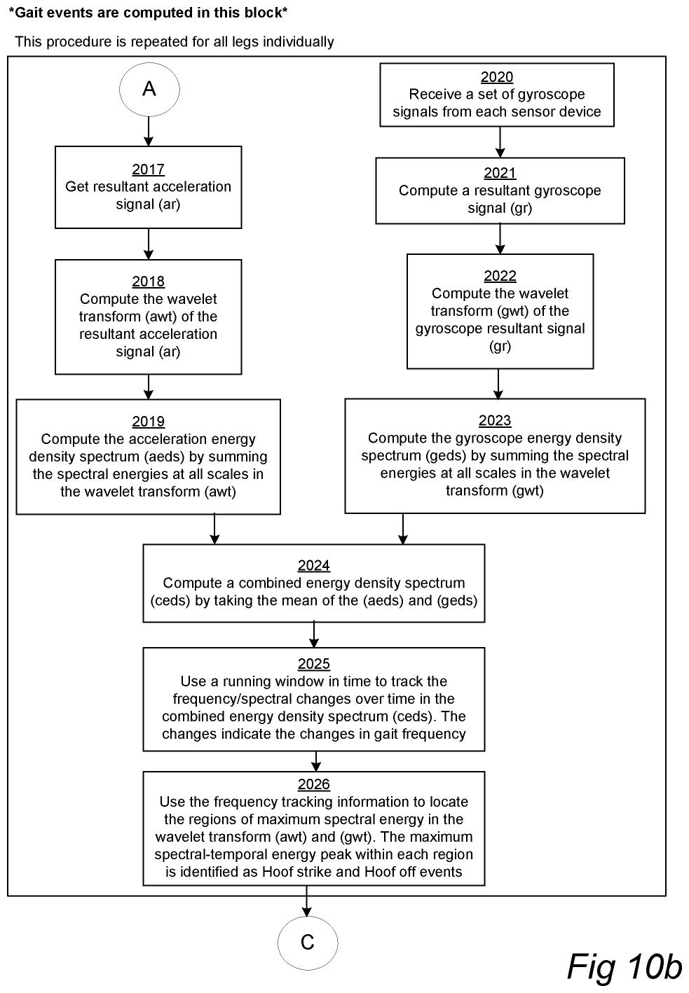

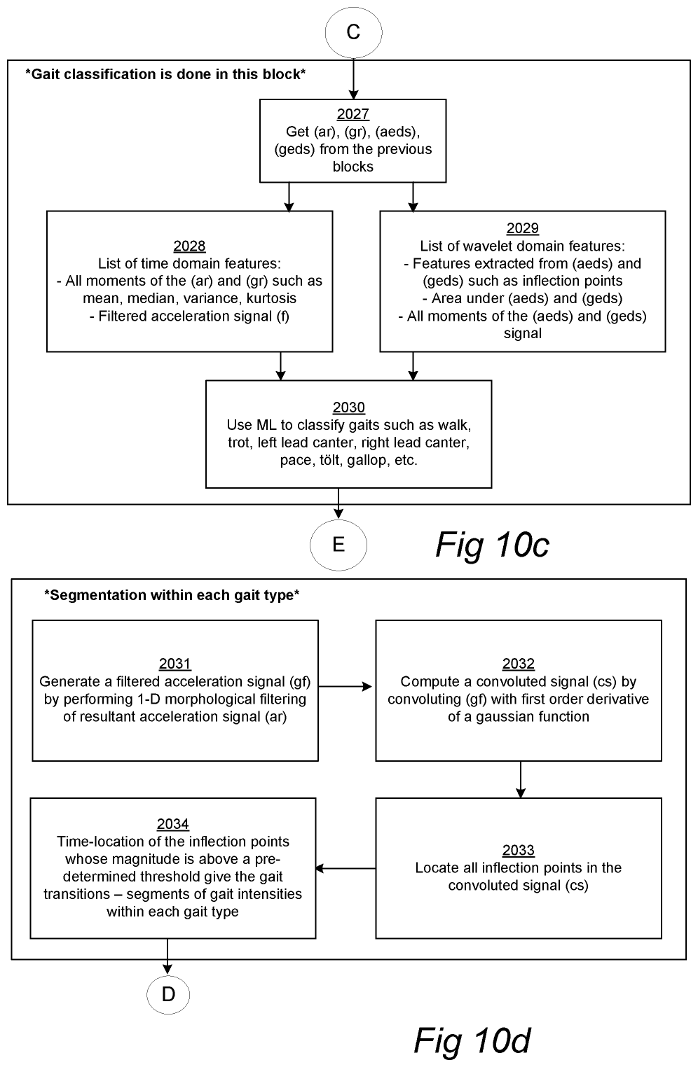

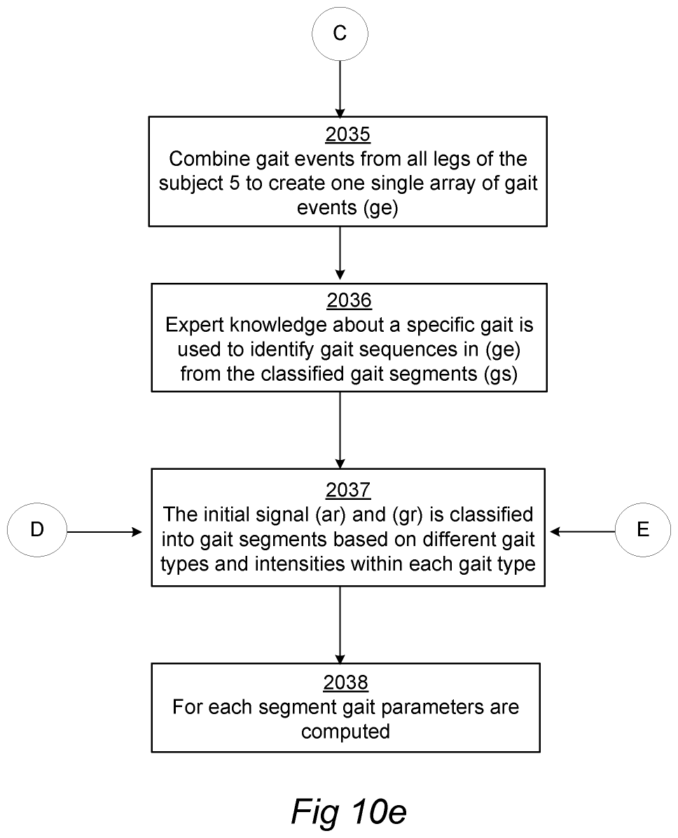

As shown in FIG. 6, the gait parameters 210 furthermore comprises one or more energy density spectrums 260. The energy density spectrums 260 are calculated based on the retrieved gait data 22a-b. Energy density spectrums 260 are used for analysing gait quality as they reveal any fluctuations in gait. Hence, the energy density spectrum(s) 260 may provide information relating to variation in gait. The energy density spectrums 260 may be assessed to detect 380 abnormalities in an equine gait of the equine animal 5. The energy density spectrums 260 and calculations thereof will be discussed thoroughly later on with reference to FIGS. 9 and 10a-e.

Some of the gait parameters 210 discussed in relation with FIG. 6 are illustrated in FIGS. 7a-h. The exemplary embodiments shown in FIGS. 7a-h are data collected from a horse. FIG. 7a shows an activity overview of one training session. In the top illustration the activity intensity 233 of the training session is shown over time, and the intensity levels are classified as zero, low, medium and high. In the second top illustration, the speed 211 is illustrated over time. The third top illustration shows the stride frequency 253, shows as strides/second over time. The lowest illustration in FIG. 7a shows the stride length 252, measures in metres over time.

FIG. 7b shows the offsets 222 for the trot gait of the horse and the average forces 216 experienced in a gait cycle for each leg. The balance 226 for a selected segment is shown by a comparison of the overall force profile with respect to the right and left side of the horse and the front and hind side of the horse.

FIG. 7c illustrates the beats 220 and beats score 221 for a selected segment, as well as the beats deviation 221b for a selected segment. As previously mentioned, the rhythm 217 is described by the consistency of beats during time laps.

In one embodiment, for trot gait, two beats 220 are used; BEAT 1, BEAT 2. In this embodiment, each beat 220 comprises information from a pair of diagonal horse legs. The first beat “BEAT 1” comprises information from the HL and FR leg. The second beat “BEAT 2” comprises information from the FL and HR leg. As shown in FIG. 7c, the deviation from the beat between the HL and FR leg can be analysed. In this example, the first beat “BEAT 1” has a beats score of 38.8% and a time of 0.26 s. The second beat “BEAT 2” has a beats score of 61.2% and a time of 0.41 s. The perfect symmetry/beats score for trot is 50%-50%.

FIG. 7d illustrates a duty factor 254 as well as the stance time 256 and swing time 255 for a selected segment. This is illustrated for each leg of the horse. For example, for the front left leg the swing time is 0.48s and the stance time is 0.19s. The duty factor is 28.4% and the stride time is 0.67s. In one embodiment, the average cadence 213 is approximately 89 strides/minute.

FIG. 7e illustrates the rhythm 217 of a horse that is walking. FIG. 7f also illustrates a walking horse, and shows the beats score 221 in a percentage, the percentage of deviation 221b from a perfect beats score/perfect symmetric beats, and an overall beat deviation score/rhythm score 221c for all steps.

FIG. 7g illustrates the rhythm 217 of a horse that is trotting. FIG. 7h also illustrates a trotting horse, and shows the beats score 221 in a percentage, the percentage of deviation 221b from a perfect beats score/perfect symmetric beats, and an overall beat deviation score/rhythm score 221c for all steps.

The analysis may include analysing the magnitude of frequency/rhythm over time for the different legs (FL, FR, HL, HR) either alone or in combination. Hence, the magnitude of frequency/rhythm can be analysed for one left and right leg alone, both left legs, both right legs, one left leg with one right leg, as well as all legs together. In this way it is possible to determine that the equine animal 5 is limping or if it has a normal gait rhythm 217 and speed 211. The analysis may further include analysing the cadence (strides/minute) at a specific step as well as over time.

The different analyses described above may be performed for walking, trotting, and/or canter or other kinds of gait.

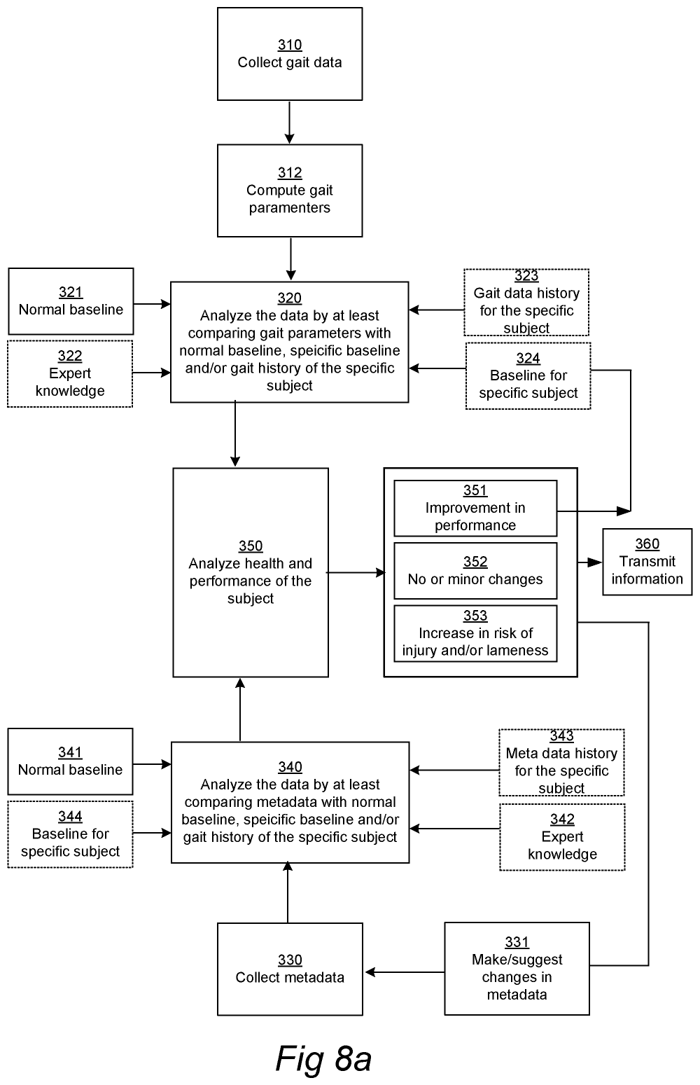

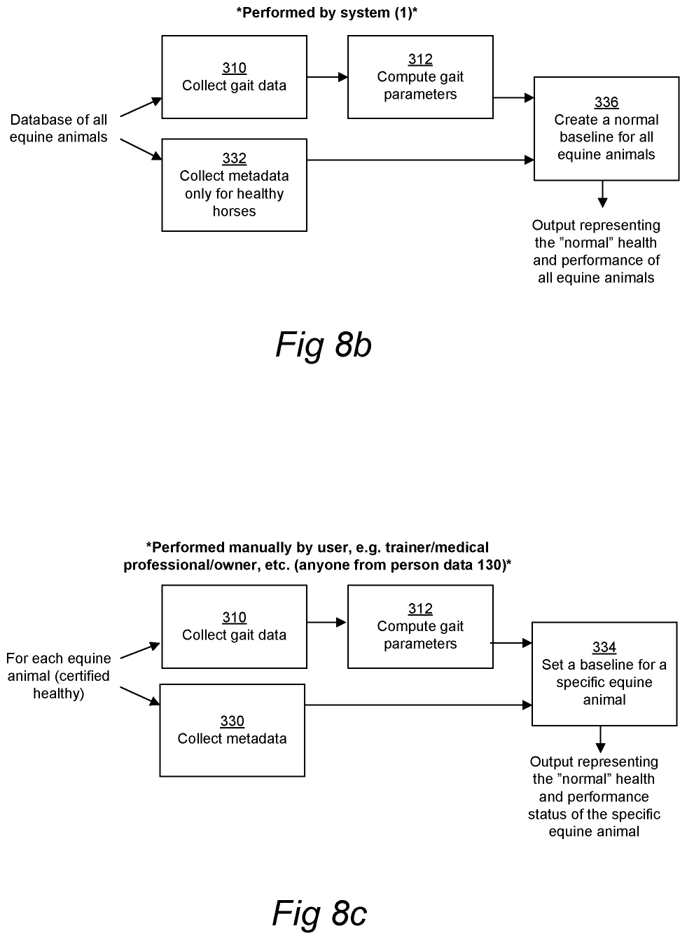

FIG. 8a-g illustrate the general provisions on how to compute different parameters regarding the health and performance of an equine animal and how this information is used to improve performance and to detect increase in risk of injury and/or detect lameness.

In a first step 310, gait data are collected from the gait sensor devices 20a-d. In a further step 312, one or more gait parameters are computed using the collected gait data. As has already been described, the gait parameters 210 may also be computed by combining metadata 110 and gait data 22a-d. In one embodiment, although not illustrated, some of the gait parameters 210 can be assessed only using metadata 110.

In a next step 320, gait parameters 210 are compared against a normal baseline 321. If available, the gait parameters 210 may further be compared against gait data 22a-d history for the specific subject 323 or compare the gait parameters 210 against a baseline for the specific subject. If available, the parameters are also analyzed 320 by inputting expert knowledge 322.

Metadata 110 is/are collected in step 330. In a next step 340, metadata 110 are compared against a normal baseline 341. If available, the metadata 110 may further be compared against metadata history for the specific subject 343 or compare the metadata 110 against a baseline for the specific subject. If available, the data is also analyzed 340 by inputting expert knowledge 342.

The analyzed data from the gait parameters 210 and metadata 110 (as analysed in steps 320 and 340) are used to analyze 350 health and performance of the subject. As described with relation to FIGS. 4a-b, this may be computed by a sensor controller 90, a web-based application programming interface 70, a storage unit 12, a computing unit or by an external device 50. The analyzed health and performance data 350 is used to determine if one can see improvement(s) in performance 351, if there is no or at least one minor change 352 in the gait quality, performance or health and/or if there is an increase in risk of injury and/or lameness 353 of the subject 5. If the data indicates increase risk of injury and/or lameness 353, the system may compute suggestions relating to changes 331 in the metadata 110 that would be beneficial. The analyzed health and performance data 350 and its findings is preferably transmitted 360 to a user. The information may be transmitted to an interface of the external device 50.

The findings 351, 352, 353 may be used to evaluate information on a short-term or long-term perspective. The findings 351, 352, 353 on the short-term 308 and/or long-term 309 perspective may be used to rank the quality of horses, rank the quality of stables and/or to rank the quality of some of the metadata 110. Metadata 110 that could be ranked is for example the accessory data 140, quality of service of a person, e.g. the person data 130, and/or the effectiveness of the training regime, e-g. the training data 150. It may for example be beneficial to rank the quality of a horse before competitions and/or during a buying/selling process. It may for example be beneficial to rank an accessory data 140 in order to determine which saddle that has the lowest or best impact on the gait of the horse.

FIG. 8b illustrates how the normal baseline 321 is created using a database of many equine animals. In a first step, the gait data 22a-d of healthy subjects are collected 310, and the gait parameters 210 are computed 312 for the subjects. Moreover, metadata 110 is collected 332 from healthy horses. The information from both the computed gait parameters 210 and the collected metadata 110 is used to create 336 a normal baseline for all equine animals. The output from creating a normal baseline for all equine animals represents the normal health and performance status of an equine animal. This evaluation is performed by the system 1 itself.

FIG. 8c illustrates how to set a baseline for a specific equine animal. Gait data 22a-d is collected 310, and gait parameters 210 are computed 312. Moreover, metadata 110 is collected. The computed gait parameters 210 and the metadata 110 is used to set 334 a baseline for a specific equine animal, whose output will then represent the normal health and performance status of that specific equine animal. The process described in FIG. 8c is preferably performed manually by an expert such as a trainer, medical professional, owner or similar, i.e. anyone from the person data 130.

In some embodiments, the computing unit 10 is configured to compute a total health score and/or risk of injury and lameness score based on at least one metadata 110 and at least one gait parameter 210. The total score may be computed with no weight factor or may be computed using one or more weight factor. Weight factors are not needed if the different parameters/data are regarded as having the same importance, but may be beneficial if one or more of the gait analysis parameters are considered more important than others. In one embodiment the total score is a weighted average of at least one gait parameter 210 and one metadata 110. The total score may be used to either determine gait quality, health or performance compared to the subject 5 itself, or compared to the reference group data. The computing unit 10 may further be configured to rank the total scores of all analysed subjects 5 to generate a comprehensive list of the assessments.

The method of collecting and analysing the gait data 22a-d will now be described with reference to FIG. 9. The plurality of sensor devices 20a-d collect 310 sensor data/gait data 22a-d. For each sensor 20a-d, the system 1 collects the gait data 22a-d. From the gait data 22a-d one or more gait parameters 210 will be computed 312. In one embodiment, the gait parameters 210 comprises sets of acceleration signals and a gyroscope signals. In one embodiment, the gait parameters 210 comprises sets of acceleration signals, gyroscope signals and possibly also magnetometer signals.

The system then computes 370 if the data corresponds to a gait related activity or rest/inactive state by comparing 372 the data with predefined thresholds 374. If it is determined that the subject is in an active state, the system 1 computes 376, 378 an acceleration energy density spectrum 260 as well as a gyroscope energy density spectrum 260 using magnitude of the resultant acceleration and gyroscope signal obtained from each individual axes. If no active state is determined, the system 1 may collect 310 additional gait data 22a-d and rerun the process according to FIG. 9. The computed gyroscope and acceleration energy density spectrums 260 are valuable for analysing gait quality as they reveal any fluctuations in gait. The energy density spectrums may be assessed to detect 380 abnormalities in an equine gait of the equine animal 5.

Gait abnormalities of the horse may include lameness, as well as a variety of neuromuscular disorders such as shivers, stringhalt, shoulder slips, tetanus or dropped elbows. Additionally, gait abnormalities may be associated with the musculotendinous unit, including abnormalities such as rhabdomyolysis, fibrotic myopathy, peroneus tertius rupture, upward fixations of the patella or flexor tendon and suspensory ligament ruptures. Any of these abnormalities may in some aspect affect the gait of the equine animal 5. By for example analysing the energy density spectrums, cause, effect and possible remedies may be discovered.

Detecting 380 abnormalities in an equine gait of the equine animal 5 involves either comparing the energy density spectrums from the acceleration and the gyroscope from each individual leg and/or by combining the energy density spectrums from the acceleration and the gyroscope to a combined energy density spectrum. The changes in gait speed and gait classification (type of gait) lead to changes in spectral energies in the individual acceleration and gyroscope energy density spectrums and the combined energy density spectrum of the four legs of the equine animal 5. The system uses a moving window in time to track these changes in spectral energy to setup spectral-temporal boundaries. The maximum spectral-temporal energy peak within each boundary is identified as Hoof strike and Hoof off events. Once the gait event has been determined for one leg, all gait events from all legs are combined to create one single array of gait events. Expert knowledge about a specific gait may be used to identify gait sequences in gait events to further improve the classification of different gaits.

In addition, the gait data 22a-d collected 310 from sensor devices 20a-d may be combined with metadata 110 in order to compute 312 some of the gait parameters 210. In one example gait data 22a-d is combined with GPS data. In such embodiment, the GPS-signals are combined with the gait data 22a-d using sensor fusion techniques such as Kalman filtering to estimate speed, velocity and stride length.

More detailed flowcharts of how to compute gait parameters 210 are illustrated in FIGS. 10a-10e and the associated method steps 2000 to 2038.

FIG. 10a illustrates how to determine if a subject 5 is performing a gait-related activity or if it is inactive. The computing unit is further configured to receive 210 a set of acceleration signals from each sensor device. For each set of received acceleration signals, a resultant acceleration signal is computed 2011. A moving std signal is then computed 2012 based on the resultant acceleration signal. A filtered acceleration signal is generated 2013 by performing 1-D morphological filtering of moving std. In a next step, if a percentage of values in the filtered acceleration signal is greater than a pre-determined activity threshold the method is continued to step 2015 where the procedure is repeated for all legs. If the step in 2014 is not fulfilled, it is determined that the subject 5 is inactive. In order to evaluate step 2014, an activity threshold may be used 2014b. In step 2016, if all legs fulfil the condition in step 2014, it is determined that the subject 5 is performing a gait-related activity. If not, it is determined that the subject is not performing a gait-related activity.

FIG. 10b illustrates how gait events are computed. The process in FIG. 10b is repeated for all legs individually. The process in FIG. 10b starts if it is determined that the subject 5 is performing a gait-related activity.

The system 1 receives the resultant acceleration signal 2017. In a next step, 2018, the wavelet transform is computed of the resultant acceleration signal. The acceleration energy density spectrum (aeds) is computed 2019 by summing the spectral energies at all scales in the wavelet transform (awt).

The system 1 receives 2020 a set of gyroscope signals from each sensor device 20a-d. A resultant gyroscope signal is computed 2021. A wavelet transform is computed 2022 of the gyroscope resultant signal. The gyroscope energy density spectrum (geds) is computed 2023 by summing the spectral energies at all scales in the wavelet transform (gwt). A combined energy density spectrum (ceds) is computed 2024 by taking the mean of the acceleration energy density spectrum (aeds) and the gyroscope energy density spectrum (geds). In step 2025, a running window in time is used to track the frequency/spectral changes over time in the combined energy density spectrum (ceds). The changes indicate the changes in gait frequency. In step 2026, the frequency tracking information is used to locate the regions of maximum spectral energy in the wavelet transform (awt) and (gwt). The maximum spectral-temporal energy peak within each region is identified as Hoof strike and Hoof off events.

FIG. 10c, illustrates gait classification. Based on the signals from the sensor devices 20a-d, the system uses time domain features 2028 and wavelet domain features 2029 to classify gait 2030, for instance whether the subject is walking, trotting, etc. The time domain features may be all moments of the acceleration signals and gyroscope signals, such as mean values, median, variance and kurtosis. The time domain feature may also include filtering the acceleration signal. The wavelet domain features may be extracted from the energy density spectrums, such as for example inflection points, area under the energy density spectrums, as well as moments of the energy density spectrum signals.

FIG. 10d illustrates segmentation within each gait type. In step 2031, a filtered acceleration signal (gf) is generated by performing 1-D morphological filtering of resultant acceleration signal (ar). In step 2032, a convoluted signal (cs) is computed by convoluting (gf) with first order derivative of a gaussian function. All inflection points in the convoluted signal is located 2033. In step 2034, the time-location of the inflection points whose magnitude is above a pre-determined threshold gives the gait transitions, i.e. segments of gait intensities within each gait type.

In FIG. 10e, all gait events from all legs of the subject 5 are combined 2035 to create one single array of gait events. Expert knowledge about a specific gait may be used 2036 to identify gait sequences in (ge) from the classified gait segments (gs). In step 2037, the initial signal (ar) and (gr) are classified into gait segments based on different gait types and intensities within each gait type. For each segment, gait parameters are computed 2038.

In one embodiment, the computing unit 10 is further configured to compute statistical data of at least one the gait parameters 210 and/or metadata 110. Statistical data may be used to more accurately assess future health and performance assessment of new or previously encountered subjects 5. In this regard, the computing unit 10 further comprises self-learning features. For instance, the system may perform autonomous classifications based on previously analysed gait patterns. The training dataset used by the computing unit 10 preferably comprises the reference group data and/or individual previously generated assessments of the specific gait analysis subject 5. The classifications may relate to one or more of the disorders discussed above, and the classifications are preferably made based on the one or more metadata 110 and/or one or more gait parameters 210. To perform the classifications and thus more accurately determine a gait quality, the computing unit 10 may implement binary, multi-class, or multi-label classification and/or clustering algorithms. For instance, algorithms such as logistic regression, support vector machines, kernel estimation, decision trees and/or artificial neural networks may be utilized. Upon accurately or inaccurately having determined a gait quality, the learning parameters are used for subsequent training of the algorithm to improve its accuracy.

For the computed data discussed above, the storage unit 12 may be configured to store the statistical data, the gait pattern indices and the health and performance assessment. Further, the storage unit 12 may further be configured to transmit this data to the external device 50.

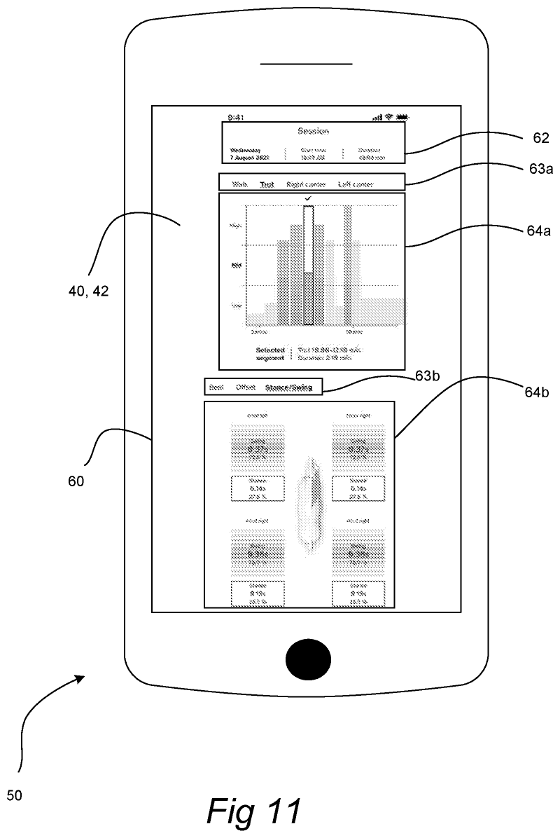

Upon having received any of the data transmitted by the storage unit 12, the external device 50 is configured to present information to the user of the external device on the display 60. This is illustrated in FIG. 11. The external device 50 is configured to present one or more of metadata 110, gait parameters 210 and/or the final assessment regarding health and performance.

The presentation of information is preferably done using any comprehensive graphical user interface being directly intractable via the display 60 by the user of the external device 50. The information may be retrieved as a request from the external device 50 to the storage unit 12. The information may also be transmitted in real-time.

In an embodiment of the invention, the computing unit 10 is further configured to generate and transmit a deviating signal indicating that something or some data in the graphs/parameters/data is abnormal. The deviating signal may be generated as a result of a detected value greatly diverging from an expected value relating any of the parameters of the assessment. For instance, if an essential classification which requires immediate attention has been made, this may be transmitted to the external device 50. Consequently, the external device 50 is configured to present said received deviating signal to the user. Furthermore, the deviating signal may also be broadcasted to many devices if necessary. A deviating report of the cause of the deviating signal may also be generated and transmitted to the external device 50. The external feedback may be in the form of a sound, vibrations, text message, e-mail, phone call, etc.

In FIG. 11, an illustration of an external device 50 is shown. More specifically, the display 60 of an external device 50 is depicted. The display 60 may be configured to present any type of information being produced by, associated with or in some sense related to the system 1. Accordingly, performance and health of different entities (subjects, trainers, riders, etc.) may be viewed in the display 60.

The display 60 preferably comprises a graphical user interface (GUI), such as the one shown in FIG. 11. The GUI may comprise an upper tab 62 comprising general information of what type of information is being currently presented on the display 60. In the shown example, the upper tab 62 describes that the presented information is related to a particular training session, i.e. that the GUI comprises information related to the training data 150. Moreover, a particular date, start time and duration is shown. The upper tab 62 may alternatively describe that the GUI presents other types of metadata 110 or gait parameters 210, such as subject data 120, person data 130, accessory data 140, stride details, or activity details 230.

The GUI may further comprise a menu tab 63a-b wherein the user of the device 50 may switch between specific information related to the current e.g. training session. FIG. 11 currently shows that the user has selected to view data related to trot in a first information box 64a and data related to Stance/Swing in a second information box 64b. The GUI may comprise any number of simultaneously active information boxes 64a-b such that the user may customize its appearance based on interest.

The information presented in the GUI of the display 60 of the external device 50 can for example show information related to different training routines; the subject's 5 movement over slopes; the subject's 5 movement clockwise around a lap; the subject's 5 movement anti-clockwise around a lap; and so forth. Accordingly, the display 60 may indicate how the subject 5 is acting when walking in a straight line, or trotting in lunges in clockwise or anti-clockwise direction, respectively. The information presented in the GUI may be viewed for any number of subjects 5, trainers and/or riders, simultaneously (e.g. in different information boxes 64a-b) or one by one.

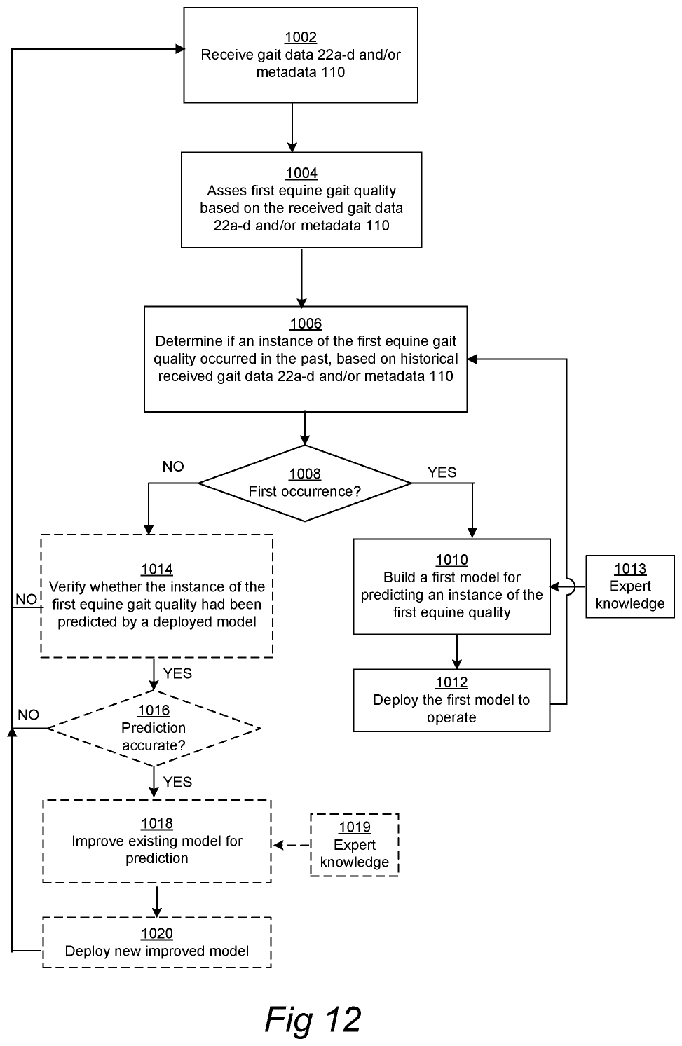

One embodiment of a method of predicting equine gait quality of an equine animal is illustrated in FIG. 12. The method preferably comprises receiving, 1002, gait data 22a-d, and/or metadata 110 as has previously been described. The method further comprises assessing, 1004, a first equine gait quality based on the received gait data 22a-d and/or metadata 110; and then determining, 1006, 1008 if an instance of the first equine gait quality occurred in the past, based on historically received gait data 22a-d and/or metadata 110.

If this is a first occurrence of the first equine gait quality, 1008-yes, then based on gait data 22a-d and/or metadata 110 received before detecting the first equine gait quality and building a first model for predicting an instance of the first equine gait quality. Once the first model is built 1010, the first model is deployed to operate, 1012. The first model may be built using expert knowledge 1013 as input. If this is not a first occurrence of said first equine gait quality, 1008-no, the method comprises verifying, 1014, whether this instance of the first equine gait quality had been predicted by a deployed model for predicting an instance of the first equine gait quality. If the instance of the first equine gait quality had not been predicted by the deployed model for predicting an instance of the first equine gait quality, or the prediction was not accurate, step 1016-no, the method comprises developing an improved existing model for predicting an instance of the first equine gait quality and deploying 1020 said improved model to operate. Export knowledge may be used as input 1019 in order to improve the existing model. In a preferred embodiment, the operation of developing another model for predicting an instance of the first equine gait quality may comprise re-training the first model on a new set of gait data 22a-d and/or metadata 110.

Preferably, the method may further comprise determining if in the received gait data 22a-d and/or metadata 110 one or more equine gait quality coincide with the first equine gait quality and then use the gait data 22a-d and/or metadata 110 being indicative of the one or more equine gait quality coinciding with the first equine gait quality to build the first model for predicting an instance of the first equine gait quality. Hereby, additional influencing factors (apart from the gait data 22a-d and/or metadata 110 used to detect the equine gait qualities) are used to develop (build) the prediction model to improve its accuracy of prediction.

In yet another alternative embodiment the method according to embodiment the method comprises clustering at least some of the received time series of the gait data 22a-d and/or metadata 110; into at least one cluster and then using the time series of gait data 22a-d and/or metadata 110; from the at least one cluster for building the first model for predicting an instance of said first equine gait quality.

This embodiment further improves accuracy of the prediction model because it exploits relationships between the gait data 22a-d and/or metadata 110 that led to detection of the equine gait quality and other time series of the gait data 22a-d and/or metadata 110. The relationships between the time series in a cluster are not only temporal but may also be of a different nature (e.g. based on temperature at the location where the equine animal is located or physical location, etc.). Thus it is possible to detecting trends in at least some of the time series of gait data 22a-d and/or metadata 110 that are indeed related with the first equine gait quality but occur prior to the first equine gait quality. This, in turn, allows for more accurate prediction of equine gait qualities.

In a further preferred embodiment, the received gait data 22a-d and/or metadata 110 comprise gait data 22a-d and/or metadata 110 received as individual values and the method comprises converting the individual values to time series of values. In one exemplary embodiment the computing unit 10 is further configured to build a model using the received gait data 22a-d and/or metadata 110 and deploying the model for predicting of equine gait quality. In one exemplary embodiment the computing unit 10 is configured to receive gait data 22a-d and/or metadata 110 received as time series of values representing gait characteristics and/ or metadata 110 associated with the equine animal.

The computing unit 10, is also operative to detect a first equine gait quality for the equine animal and determine if an instance of the first equine gait quality occurred in the past based on historical gait data 22a-d and/or metadata 110. If this is a first occurrence of the first equine gait quality, then based on gait data 22a-d and/or metadata 110 received before detecting the first equine gait quality, the computing unit 10, is operative to build a first model for predicting an instance of said first equine gait quality and then deploy the first model in the to operate.

In a preferred embodiment to develop another model for predicting an instance of the first equine gait quality the computing unit 10 is operative to re-train the first model on a new set of gait data 22a-d and/or metadata 110. In yet another preferred embodiment to develop another model for predicting an instance of the first equine gait quality the apparatus is operative to update the first model.

Preferably, the computing unit 10, is further operative to determine if in the received gait data 22a-d and/or metadata 110 one or more equine gait qualities coincide with the first equine gait quality and use the received gait data 22a-d and/or metadata 110 indicative of the one or more equine gait qualities coinciding with the first equine gait quality for building the first model for predicting an instance of the first equine gait quality.

Preferably, the computing unit 10, is further operative to cluster at least some of the received gait data 22a-d and/or metadata 110 into at least one cluster and use the time series of the gait data 22a-d and/or metadata 110 from the at least one cluster for building the first model for predicting an instance of the first equine gait quality.

In a preferred embodiment the received gait data 22a-d and/or metadata 110 data received as individual values and the computing unit 10, is operative to convert the individual values to time series of values.

The advantages of the present solution include (but are not limited to) the following: Equine gait qualities are predicted before they occur, and remedial measures are taken to avoid equine gait qualities that can be harmful to the equine animal. This enables a proactive approach of autonomous equine gait quality management compared to the current reactive approach. Data e.g. gait data 22a-d and/or metadata 110 are autonomously determined for the incident/anomaly rather than purely relying on historical knowledge base and/or equine expertise. Autonomous recommendation becomes possible due to discovery of determining impacting factors of equine gait quality. When the impacting factors are known then recommending solutions is feasible and can be derived from knowledge of how to impacting factors influence the equine gait quality.

The present disclosure provides a solution for equine gait quality prediction using a model developed by a machine learning algorithm in which the machine learning algorithm uses historical gait data 22a-d and/or metadata 110 for training. Once the model is ready, it is deployed and operates on incoming gait data 22a-d and/or metadata 110.

Accuracy of prediction of equine gait quality by the model is verified in order to improve the model and achieve higher accuracy of prediction. The amount of historical gait data 22a-d and/or metadata 110 increase as the data is collected, so if prediction is not accurate enough (e.g. gets less accurate than in previously) the machine learning algorithm re-trains on new (and in some embodiments bigger set of data) to develop an improved model for equine gait quality. If a new equine gait quality is detected (i.e. a new type of equine gait quality) the machine learning algorithm develops a model in run time for predicting instances of this newly observed equine gait quality. In a preferred embodiment there are different models deployed for predicting different types of equine gait quality (e.g. incidents related to health of the equine animal, performance etc.).

Using the initial equine gait quality that led to detection of an equine gait quality and any additional equine gait parameters and/or trends a new machine learning prediction model is built at runtime and deployed to predict future occurrence equine gait parameters. The new machine learning prediction model preferably may also be evaluated before being deployed. The evaluation may be carried out by running the model on gait data 22a-d and/or metadata 110 which, preferably, is also a set of historical gait data 22a-d and/or metadata 110 that exhibits the incident for detection of which the model has been developed, whereas the gait data 22a-d and/or metadata 110 was not used for development of the prediction model.

Also preferably, further evaluation of the prediction model is carried out in run time—the model predicts an equine gait quality and the prediction is then verified against receive gait data 22a-d and/or metadata 110. If the accuracy of the prediction is not as good as expected a new prediction model may be developed. In addition to correlation of equine gait parameters or trends in equine gait parameters to build the prediction model a cluster of time series of equine gait parameters may be used as a possible factor for prediction.

The on-demand created model can predict future equine gait quality based on historical gait data 22a-d and/or metadata 110 that can potentially help in mitigating equine gait quality before an equine gait quality problem occurring again.

The invention has been described above in detail with reference to embodiments thereof. However, as is readily understood by those skilled in the art, other embodiments are equally possible within the scope of the present invention, as defined by the appended claims.

1. An analysis system for assessing gait-related health and performance of an equine animal, the analysis system comprising:
a first sensor device arranged at a region of a first leg of the equine animal;
a second sensor device arranged at a region of a second leg of the equine animal;
a third sensor device arranged at a region of a third leg of the equine animal;
a fourth sensor device arranged at a region of a fourth leg of the equine animal;
wherein the first, the second, the third, and the fourth sensor devices each comprises at least one 3-axis accelerometer and at least one 3-axis gyroscope;
wherein the first, the second, the third, and the fourth sensor devices are configured to provide gait data; and
a computing unit configured to
receive the gait data from the first, the second, the third, and the fourth sensor devices,
receive at least one metadata associated with the equine animal,
analyze the received gait data and/or the received metadata for determining at least one gait parameter related to equine stride characteristics of the equine animal, and
analyze the at least one gait parameter and the at least one metadata to assess gait-related health and performance of the equine animal.

2. The analysis system according to claim 1, wherein the computing unit is further configured to store the assessed gait-related equine health and performance and/or to communicate the assessed gait-related equine health and performance to an external device having a display;
wherein the external device is configured to present the assessed gait-related equine health and performance to a user.

3. The analysis system according to claim 1, wherein the metadata comprises one or more of:
information of subject data of the equine animal,
information of person data of persons related to the equine animal,
information of accessory data related to accessories of the equine animal, and
information of training data of the equine animal.

4. The analysis system according to claim 1, wherein the metadata is based on data received from at least one additional sensor and/or based on data being inputted to the analysis system by a user.

5. The analysis system according to claim 1, wherein the at least one gait parameter comprises information of at least one computed energy density spectrum.

6. The analysis system according to claim 1, wherein the at least one gait parameter comprises one or more of:
information relating to stride details of the equine animal,
information relating to activity details of a training session of the equine animal, and
information relating to gait of the equine animal.

7. The analysis system according to claim 1, wherein each one of the first, the second, the third, and the fourth sensor devices further comprises a magnetometer.

8. The analysis system according to claim 1, wherein each one of the first, the second, the third, and the fourth sensor devices is arranged in one respective sensor device holder.

9. The analysis system according to claim 1, wherein the assessed gait-related equine health and performance is used to detect at least one of:
one or more improvements in performance of the equine animal,
no or at least one change in the performance of the equine animal,
no or at least one change in the health of the equine animal, and/or
an increase in risk of injury and/or lameness of the equine animal.

10. The analysis system according to claim 1, wherein the assessed gait-related equine health and performance is used to make or suggest changes in the metadata.

11. The analysis system according to claim 1, wherein the gait parameter and the metadata are analyzed by comparing them to one or more baselines and/or against historical data.

12. The analysis system according to claim 1, wherein the computing unit is further configured to:
receive a set of acceleration signals from each sensor device;
for each set of received acceleration signals, compute a resultant acceleration signal;
based on the computed resultant acceleration signals, determine if the equine animal is performing a gait related activity or is inactive; and
if it is determined that the equine animal is performing a gait related activity, compute an accelerometer energy density spectrum for each resultant acceleration signal;
wherein each accelerometer energy density spectrum corresponds to one leg of the equine animal.

13. The gait analysis systems according to claim 12, wherein determining if the equine animal is performing a gait related activity further involves:
computing a moving standard deviation signal of the resultant acceleration signals;
generating a filtered acceleration signal by performing 1-D morphological filtering of the computed moving standard deviation signal; and
determining if a total number of elements of the filtered acceleration signal having a value greater than or equal to a value of a corresponding element of a predetermined walking threshold.

14. The gait analysis system according to claim 1, wherein the computing unit is further configured to:
receive a set of acceleration signals from each sensor device;
for each set of received acceleration signals, compute a resultant acceleration signal;
compute an accelerometer energy density spectrum for each resultant acceleration signal;
wherein each accelerometer energy density spectrum corresponds to one leg of the equine animal;
receive a set of gyroscope signals from each sensor device;
for each set of received gyroscope signals, compute a resultant gyroscope signal; and
for each resultant gyroscope signal, compute a gyroscope energy density spectrum;
wherein each gyroscope energy density spectrum corresponds to one leg of the equine animal.

15. The gait analysis system according to claim 14, wherein the computing unit is further configured to combine the accelerometer energy density spectrum and the gyroscope energy density spectrum;
wherein the combined energy density spectrum is used to determine one or more gait parameters.

16. The gait analysis system according to claim 14, wherein the computing unit is further configured to analyze the computed energy density spectrums by:
measuring a variability by comparing each energy density spectrum to itself over a predetermined time period, and/or
measuring a symmetry by comparing an energy density spectrum of a left leg of the equine animal to an energy density spectrum of a right leg of the equine animal, and/or
measuring a normality by comparing each energy density spectrum to at least one energy density spectrum of a leg from a reference population group exhibiting no gait pathology.

17. The gait analysis system according to claim 1, wherein the computing unit is further configured to compute statistical data and/or historical data of at least one of the metadata and the at least one gait parameter.

18. The gait analysis system according to claim 1, wherein the computing unit is further configured to generate and transmit a deviating signal to an external device if at least one of the metadata and/or the at least one gait parameter exceeds a predetermined deviating threshold value.

19. A method for assessing gait-related health and performance of an equine animal being equipped with a first sensor device at a region of a first leg of the equine animal, a second sensor device at a region of a second leg of the equine animal, a third sensor device at a region of a third leg of the equine animal, and a fourth sensor device at a region of a fourth leg of the equine animal, wherein the first, the second, the third, and the fourth sensor devices each comprises at least one 3-axis accelerometer and at least one 3-axis gyroscope, and wherein the first, the second, the third, and the fourth sensor devices are configured to provide gait data, the method comprising:
receiving the gait data from the first, the second, the third, and the fourth sensor devices;
receiving at least one metadata associated with the equine animal;
analyzing the received gait data and/or the received metadata for determining at least one gait parameter related to equine stride characteristics of the equine animal; and
analyzing the at least one gait parameter and the at least one metadata to assess gait-related health and performance of the equine animal.
